(19) United States
(12) Patent Application Publication
(10) Pub. No.: US 2023/0173764 A1
(43) Pub. Date: Jun. 8, 2023

(54) SURGICAL SYSTEM AND METHODS OF USE

(71) Applicant: MEDTRONIC INC., Minneapolis, MN (US)

(72) Inventors: CHRISTIAN S. NIELSEN, River Falls, WI (US); Sean Chen, Plymouth, MN (US); Kasyap V. Kasyap Seethamraju, Eden Prairle, MN (US)

(73) Assignee: MEDTRONIC INC., Minneapolis, MN (US)

(21) Appl. No.: 17/545,317

(22) Filed: Dec. 8, 2021

(51) Int. Cl. B29C 65/60; A61N 1/375; B29C 65/18; B29C 65/00

(52) U.S. Cl. CPC B29C 65/601; A61N 1/37518; B29C 65/18; B29C 66/7292; B29L 2031/7534

(57) ABSTRACT

A method of forming an implant includes positioning a first mesh component of the implant within a second mesh component of the implant to form an implant assembly. The implant assembly is manipulated to join the first mesh component with the second mesh component.

### TECHNICAL FIELD

The present disclosure generally relates to anchorage devices and methods of making anchorage devices configured for anchoring an implantable medical device within a body, wherein the anchorage device comprises at least one hemostatic agent and at least one antibacterial agent that are configured to elute over time.

### BACKGROUND

Some known anchorage devices may be used to secure an implantable medical device within a body of a patient. The anchorage device and implantable medical device can be inserted into a desired location within the body of the patient. The anchorage device can be used to help anchor or support the implantable medical device to surrounding tissue. Some known anchorage devices are used to provide temporary support to tissue during a healing process. For example, some known anchorage devices can secure one portion of tissue to another portion of tissue.

Infection and bleeding are the most serious complications after surgery. The estimated increase in costs due to surgical site infections (SSIs) was $11,876 for SSIs overall ($7003 for superficial and $25,721 for deep infections). However, within the current $6 billion hemostat market, there are few products that can address this unmet need. It would therefore be desirable to stop or reduce the flow of blood at a surgical site and/or speed up the blood clotting process while anchoring the implantable medical device to tissue. This disclosure describes an improvement over these prior art technologies.

### SUMMARY

New anchorage devices and methods are provided to help anchor or support an implantable medical device to surrounding tissue. In one embodiment, in accordance with the principles of the present disclosure, a method of forming an implant comprises positioning a first mesh component of the implant within a second mesh component of the implant to form an implant assembly and manipulating the implant assembly to join the first mesh component with the second mesh component.

In some embodiments, manipulating the implant assembly comprises dispensing a plurality of stakes through the second mesh component and into the first mesh component. In some embodiments, the stakes comprise collagen. In some embodiments, the stakes comprise gelling collagen. In some embodiments, the stakes are spaced apart from one another. In some embodiments, the stakes are arranged in a pattern. In some embodiments, each of the stakes is connected to another one of the stakes such that the stakes form a continuous line. In some embodiments, the stakes extend about at least a portion of a perimeter of the implant assembly. In some embodiments, the method further comprises cooling the implant assembly after manipulating the implant assembly.

In some embodiments, manipulating the implant assembly comprises pressing an element of a heat seal band onto the implant assembly. In some embodiments, the heat seal band forms a plurality of spaced apart horizontal seals across the implant assembly. In some embodiments, the heat seal band forms a seal about at least a portion of a perimeter of the implant assembly. In some embodiments, an interface is positioned between the heat seal band and the implant assembly to facilitate release of the heat seal band from the implant assembly after the implant assembly is manipulated. In some embodiments, manipulating the implant assembly comprises using a first heat seal band to form a plurality of spaced apart horizontal seals across the implant assembly and using a second heat seal band to form a seal about at least a portion of a perimeter of the implant assembly after using the first heat seal band.

In some embodiments, manipulating the implant assembly comprises directing heat from a heat seal band onto the implant assembly to form at least one seal. In some embodiments, an interface is positioned between the heat seal band and the implant assembly.

In some embodiments, manipulating the implant assembly comprises providing a press having a base. The base includes a plurality of spaced apart rails that define channels therebetween. The base comprises a plurality of spaced apart holes extending through each of the rails and manipulating the implant assembly comprises disposing the implant assembly in the base such that the implant assembly extends into the channels and inserting sutures through the holes and the implant assembly. In some embodiments, the method comprises moving a plate of the press toward the base with the implant assembly positioned between the plate and the base to move portions of the implant assembly into the channels before inserting sutures through the holes and the implant assembly.

In one embodiment, in accordance with the principles of the present disclosure, a method of forming an implant comprises positioning a first mesh component of the implant within a second mesh component of the implant to form an implant assembly; and manipulating the implant assembly to join the first mesh component with the second mesh component, wherein the first mesh component comprises a coating having a first polymer and at least one antibacterial agent dispersed in the first polymer, wherein the second mesh component comprises a coating having a second polymer and at least one hemostatic agent dispersed in the second polymer, and wherein manipulating the implant assembly comprises dispensing a plurality of collagen stakes through the second mesh component and into the first mesh component.

In one embodiment, in accordance with the principles of the present disclosure, a method of forming an implant comprises positioning a first mesh component of the implant within a second mesh component of the implant to form an implant assembly; and manipulating the implant assembly to join the first mesh component with the second mesh component, wherein the first mesh component comprises a coating having a first polymer and at least one antibacterial agent dispersed in the first polymer, wherein the second mesh component comprises a coating having a second polymer and at least one hemostatic agent dispersed in the second polymer, and wherein manipulating the implant assembly comprises forming at least one seal by applying heat.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWINGS

The present disclosure will become more readily apparent from the specific description accompanied by the following drawings, in which:

FIG. 1 is a plan view, in part phantom, of components of a surgical system, in accordance with the principles of the present disclosure;

FIG. 2 is a perspective view of one embodiment of a component of the surgical system shown in FIG. 1, in accordance with the principles of the present disclosure;

FIG. 3 is a perspective view of the component shown in FIG. 2;

FIG. 4 is a perspective view of the component shown in FIG. 2;

FIG. 5 is a perspective view showing features of making one embodiment of a component of the surgical system shown in FIG. 1, in accordance with the principles of the present disclosure;

FIG. 6 is a perspective view showing features of making one embodiment of a component of the surgical system shown in FIG. 1, in accordance with the principles of the present disclosure;

FIG. 7 is a perspective view showing features of making one embodiment of a component of the surgical system shown in FIG. 1, in accordance with the principles of the present disclosure;

FIG. 8 is a perspective view showing features of making one embodiment of a component of the surgical system shown in FIG. 1, in accordance with the principles of the present disclosure;

FIG. 9 is a perspective view showing features of making one embodiment of a component of the surgical system shown in FIG. 1, in accordance with the principles of the present disclosure;

FIG. 10 is a perspective view showing features of making one embodiment of a component of the surgical system shown in FIG. 1, in accordance with the principles of the present disclosure;

FIG. 10A is a perspective view showing features of making one embodiment of a component of the surgical system shown in FIG. 1, in accordance with the principles of the present disclosure;

FIG. 11 is a perspective view showing features of making one embodiment of a component of the surgical system shown in FIG. 1, in accordance with the principles of the present disclosure;

FIG. 11A is a perspective view showing features of making one embodiment of a component of the surgical system shown in FIG. 1, in accordance with the principles of the present disclosure;

FIG. 11B is a perspective view showing features of making one embodiment of a component of the surgical system shown in FIG. 1, in accordance with the principles of the present disclosure;

FIG. 12 is a perspective view showing features of making one embodiment of a component of the surgical system shown in FIG. 1, in accordance with the principles of the present disclosure;

FIG. 13 is a perspective view, in part phantom, showing features of making one embodiment of a component of the surgical system shown in FIG. 1, in accordance with the principles of the present disclosure;

FIG. 14 is a perspective view showing features of making one embodiment of a component of the surgical system shown in FIG. 1, in accordance with the principles of the present disclosure;

FIG. 15 is a perspective view showing features of making one embodiment of a component of the surgical system shown in FIG. 1, in accordance with the principles of the present disclosure;

FIG. 16 is a perspective view showing features of making one embodiment of a component of the surgical system shown in FIG. 1, in accordance with the principles of the present disclosure;

FIG. 17 is a perspective view showing features of making one embodiment of a component of the surgical system shown in FIG. 1, in accordance with the principles of the present disclosure; and

FIG. 18 is a perspective view showing features of one embodiment of a component of the surgical system shown in FIG. 1, in accordance with the principles of the present disclosure.

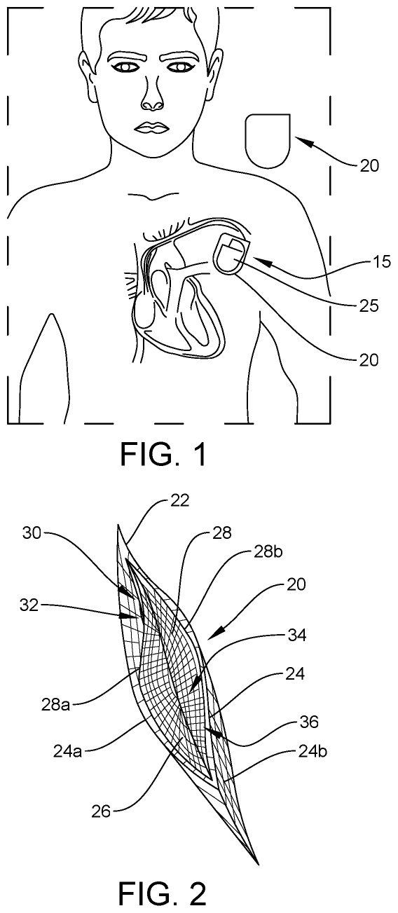

### DETAILED DESCRIPTION

For the purposes of this specification and appended claims, unless otherwise indicated, all numbers expressing quantities of ingredients, percentages or proportions of materials, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding the numerical ranges and parameters set forth herein, the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

Surgical site infections are increasing in frequency, severity and cost. Antibiotics are effective in eliminating short term infection at surgical sites. This disclosure provides a fundamental shift in the thinking of how to address infection by providing a new approach that can lead to better infection prevention outcomes, better overall healing and eliminate or minimize the use of antibiotics.

This disclosure is directed to a surgical system that includes an implant, such as, for example, an anchorage device comprising one or more active pharmaceutical ingredients together with one or more hemostatic agents in order to prevent or reduce bleeding via the one or more hemostatic agents and provide another effect, such as, for example, an antimicrobial effect via the one or more antimicrobial agents.

Time has shown that anchorage devices such as, for example the TYRX™ Absorbable Antibacterial Envelope and EZ Glide, which include mesh substrates that are coated with one or more polymers, such as, for example, one or more tyrosine-derived polyarylate and include one or more antibacterial/antimicrobial agents that are dispersed in the polymer, are effective implanted Implantable Pulse Generator (IPG) stabilization systems that have been designed with an antibacterial coating that further enhances the performance. This type of coating may also be included in other devices, such as, for example, devices for use in connection with soft tissue and devices that are sized for non-IPG applications, such as, for example the LVAD drive line and general surgery.

In order to provide a hemostatic effect, a first component, such as, for example, an anchorage device (e.g., the TYRX™ Absorbable Antibacterial Envelope or EZ Glide) can be positioned within a second component, such as, for example, a pocket or envelope that elutes one or more hemostatic agents to allow the assembly of the TYRX™ Absorbable Antibacterial Envelope (the first component) and the hemostatic device (the second component) to treat and/or prevent bacterial/microbial infection, while simultaneously reducing or preventing bleeding.

In some embodiments, the first and second components are joined using bioabsorbable “glue”, heat staking, bioabsorbable suture, pocket in pocket, etc. In some embodiments, the second component is sized to fit the first component. In some embodiments, the second component may be oversized, smaller than, or a separate envelope the first component would slip into.

In some embodiments, a robot dispenses stakes down the second component using gelling collagen and the assembly of the first and second components is then cooled after the stakes are dispensed. In some embodiments, robotic actuation distributes a selective patten of droplets of a collagen rich solution, which define the stakes. Both partial and droplet distribution are contemplated. In some embodiments, the stakes comprise a UV curable solution. In some embodiments, the stakes are in the form of edge staking or a continuous bead. In some embodiments, if exact sizing of the second component was not employed, excess martial is laser or die punch trimmed.

In some embodiments, a press presents a flash heat band onto the top of the second component to join the second component to the first component. In some embodiments, the heat flash band bonds the second component to the first component. In some embodiments, the heat flash band is selectively applied to one or more portions of the assembly of the first and second components. In some embodiments, the heat flash band extends along at least a portion of a perimeter of the assembly of the first and second components. In some embodiments, the heat flash band is modulated in and out of the plane of the first component providing a spot staking in defined locations. In some embodiments, a Kapton tape interface is maintained between the heat seal band and the second component to facilitate release after partial cooling. In some embodiments, the heat flash band, if exact sizing of the second component was not employed, excess martial is laser or die punch trimmed.

In some embodiments, a small press presents a flash heat band onto the second component bonding the second component onto the first component. In some embodiments, the heat flash band is selectively applied to one or more portions of the assembly of the first and second components. In some embodiments, the heat flash band extends along at least a portion of a perimeter of the assembly of the first and second components. A similar process could bond the edge perimeter in the same step. In some embodiments, the heat seal band is formed over a small post or the like presenting a spot stake weld. In some embodiments, a Kapton tape interface is maintained between the heat seal band and the second component. In some embodiments, the excess martial is laser trimmed.

In some embodiments, a small press is presented to the second component to deforming the second component downward in a corrugated manner along with the first component that has been placed over the thin, lower mandrel. A needle inserted through holes of the press and the assembly of the first and second components such that a suture that is attached to the needle is pushed through the assembly of the first and second components to join the second component with the first component using specific (quilting) stich positions. In some embodiments, if exact sizing of the second component is not employed, excess martial is laser or die punch trimmed.

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

This disclosure is directed to a surgical system 15. In some embodiments, system 15 includes one or more implant assemblies, such as, for example, an anchorage device 20. In some embodiments, the components of anchorage device 20 can be fabricated from biologically acceptable materials suitable for medical applications, including metals, synthetic polymers, allografts, xenografts, isografts, ceramics and bone material and/or their composites, depending on the particular application and/or preference of a medical practitioner. For example, the components of anchorage device 20, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, tyrosine polyarylate, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polylactide, polyglycolide, polytyrosine carbonate, polycaroplactone, polytrimethelene carbonate, polydioxanone, polyhydroxyalkanoates and their combinations.

Various components of anchorage device 20 may have material composites, including the above materials, to achieve various desired characteristics such as strength, rigidity, elasticity, compliance, biomechanical performance, durability and radiolucency or imaging preference. The components of anchorage device 20, individually or collectively, may also be fabricated from a heterogeneous material such as a combination of two or more of the above-described materials. The components of anchorage device 20 may be monolithically formed, integrally connected or include fastening elements and/or instruments, as described herein.

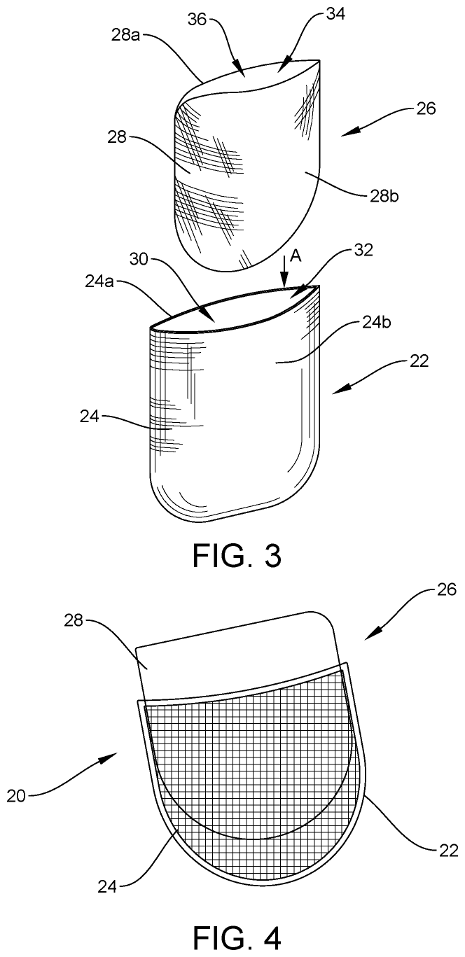

Anchorage device 20 includes a first component 22 comprising a first substrate 24 and a second component 26 comprising a second substrate 28. In some embodiments, first substrate 24 is a pocket or envelope in which second component 26 and/or an implantable medical device, such as, for example, a medical device 25 can be at least partially disposed. That is, substrate 24 is a pouch, bag, covering, shell, or receptacle. For example, substrate 24 can include a first piece 24a and a second piece 24b that is joined with first piece 24a. First and second pieces 24a, 24b are joined to form the pocket or envelope. In some embodiments, first and second pieces 24a, 24b are joined along three sides of the pocket or envelope to form a cavity 30, as shown in FIG. 3, for example. First and second pieces 24a, 24b are not joined at a fourth side of the pocket or envelope to define an opening 32 such that an implantable medical device, such as, for example, device 25 and/or component 26 can be inserted through opening 32 and into cavity 30 to enclose, encase or surround all or a portion of the implantable medical device and/or component 26 within cavity 30. In some embodiments, component 26 is inserted into cavity 30 by moving component 26 in the direction shown by arrow A in FIG. 3 through opening 32 and into cavity 30. In some embodiments, first and second pieces 24a, 24b are joined with one another along three sides of the pocket or envelope by heat, ultrasonically, bonding, knitting, or adhesive. In some embodiment, the pocket or envelope is monolithically formed by molding the pocket or envelope or producing the pocket or envelope by 3D printing, for example.

In some embodiments, first and second pieces 24a, 24b are portions of a single sheet that is bent to produce a fold at one end of the pocket or envelope. First and second pieces 24a, 24b are joined along sides of the pocket or envelope that extend transverse to the fold such that the fold and the sides of the pocket or envelope do not have any openings. First and second pieces 24a, 24b are not joined at an end of the pocket or envelope opposite the fold to define opening 32 at the end such that medical device 25 and/or component 26 can be inserted through opening 32 and into cavity 30, which is defined by inner surfaces of first and second pieces 24a, 24b.

In some embodiments, first and second pieces 24a, 24b each include a mesh. In some embodiments, first piece 24a includes a mesh including pores having a first size and second piece 24b includes a mesh including pores having a second size, wherein the first size is different than the first size. In some embodiments, the first size is greater than the second size. In some embodiments, the first size is less than the second size.

In some embodiments, first and second pieces 24a, 24b are formed from the same material. In some embodiments, one of first and second pieces 24a, 24b is formed from a first material, such as, for example, one or more of the materials discussed herein, and the other one of first and second pieces 24a, 24b is made from a second material, such as, for example, one or more of the materials discussed herein, wherein the second material is different than the first material. For example, first piece 24a may be formed from a biodegradable and/or bioresorbable material and second piece 24b may be formed from a non-biodegradable and/or non-bioresorbable material, or vice versa. In some embodiments, first and second pieces 24a, 24b are each formed from a biodegradable and/or bioresorbable material, wherein the biodegradable and/or bioresorbable materials degrade and/or resorb at the same rate. In some embodiments, first and second pieces 24a, 24b are formed from different biodegradable and/or bioresorbable materials, wherein one of the biodegradable and/or bioresorbable materials degrades and/or resorbs more quickly than the other biodegradable and/or bioresorbable material.

In some embodiments, first and second pieces 24a, 24b each include a single layer of material, such as, for example, one or more of the materials discussed herein. In some embodiments, at least one of first and second pieces 24a, 24b includes multiple layers. In some embodiments, the multiple layers include more than one layer of a mesh.

In some embodiments, second substrate 28 is a pocket or envelope in which an implantable medical device, such as, for example, device 25 can be at least partially disposed. That is, substrate 28 is a pouch, bag, covering, shell, or receptacle. For example, substrate 28 can include a first piece 28a and a second piece 28b that is joined with first piece 28a. First and second pieces 28a, 28b are joined to form the pocket or envelope. In some embodiments, first and second pieces 28a, 28b are joined along three sides of the pocket or envelope to form a cavity 34, as shown in FIG. 3, for example. First and second pieces 28a, 28b are not joined at a fourth side of the pocket or envelope to define an opening 36 such that an implantable medical device, such as, for example, device 25 can be inserted through opening 36 and into cavity 34 to enclose, encase or surround all or a portion of the implantable medical device within cavity 34. In some embodiments, device 25 is inserted into cavity 34 by moving device 25 in the direction shown by arrow A in FIG. 3 through opening 36 and into cavity 34. In some embodiments, first and second pieces 28a, 28b are joined with one another along three sides of the pocket or envelope by heat, ultrasonically, bonding, knitting, or adhesive. In some embodiment, the pocket or envelope is monolithically formed by molding the pocket or envelope or producing the pocket or envelope by 3D printing, for example.

In some embodiments, first and second pieces 28a, 28b are portions of a single sheet that is bent to produce a fold at one end of the pocket or envelope. First and second pieces 28a, 28b are joined along sides of the pocket or envelope that extend transverse to the fold such that the fold and the sides of the pocket or envelope do not have any openings. First and second pieces 28a, 28b are not joined at an end of the pocket or envelope opposite the fold to define opening 36 at the end such that medical device 25 can be inserted through opening 36 and into cavity 34, which is defined by inner surfaces of first and second pieces 28a, 28b.

In some embodiments, first and second pieces 28a, 28b each include a mesh. In some embodiments, first piece 28a includes a mesh including pores having a first size and second piece 28b includes a mesh including pores having a second size, wherein the first size is different than the first size. In some embodiments, the first size is greater than the second size. In some embodiments, the first size is less than the second size.

In some embodiments, first and second pieces 28a, 28b are formed from the same material. In some embodiments, one of first and second pieces 28a, 28b is formed from a first material, such as, for example, one or more of the materials discussed herein, and the other one of first and second pieces 28a, 28b is made from a second material, such as, for example, one or more of the materials discussed herein, wherein the second material is different than the first material. For example, first piece 28a may be formed from a biodegradable and/or bioresorbable material and second piece 28b may be formed from a non-biodegradable and/or non-bioresorbable material, or vice versa. In some embodiments, first and second pieces 28a, 28b are each formed from a biodegradable and/or bioresorbable material, wherein the biodegradable and/or bioresorbable materials degrade and/or resorb at the same rate. In some embodiments, first and second pieces 28a, 28b are formed from different biodegradable and/or bioresorbable materials, wherein one of the biodegradable and/or bioresorbable materials degrades and/or resorbs more quickly than the other biodegradable and/or bioresorbable material.

In some embodiments, first and second pieces 28a, 28b each include a single layer of material, such as, for example, one or more of the materials discussed herein. In some embodiments, at least one of first and second pieces 28a, 28b includes multiple layers. In some embodiments, the multiple layers include more than one layer of a mesh.

Anchorage device 20 is configured to be coupled to and/or applied to a device, such as, for example, medical device 25. In some embodiments, medical device 25 is an implantable medical device, as discussed herein. In some embodiments, medical device 25 is a non-implantable medical device, as discussed herein. In some embodiments, substrate 28 is configured to surround and/or enclose at least a portion of medical device 25, as discussed herein. Anchorage device 20 is configured to be secured to tissue to support one or more devices 25, such as grafts (e.g., abdominal aortic aneurysm grafts, etc.), stents, catheters (including arterial, intravenous, blood pressure, stent graft, etc.), valves (e.g., polymeric or carbon mechanical valves,), embolic protection filters (including distal protection devices), vena cava filters, aneurysm exclusion devices, artificial hearts, cardiac jackets, and heart assist devices (including left ventricle assist devices), implantable defibrillators, subcutaneous implantable defibrillators, implantable monitors, for example, implantable cardiac monitors, electrostimulation devices and leads (including pacemakers, lead adapters and lead connectors), implanted medical device power supplies, peripheral cardiovascular devices, atrial septal defect closures, left atrial appendage filters, valve annuloplasty devices, mitral valve repair devices, vascular intervention devices, ventricular assist pumps, and vascular access devices (including parenteral feeding catheters, vascular access ports, central venous access catheters).

Device 25 may also include, for example, surgical devices such as sutures of all types, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps. Device 25 may also include, for example, orthopedic devices such as joint implants, acetabular cups, patellar buttons, bone repair/augmentation devices, spinal devices (e.g., vertebral disks and the like), bone pins, cartilage repair devices, and artificial tendons. Device 25 may also include, for example, dental devices such as dental implants and dental fracture repair devices. Device 25 may also include, for example, drug delivery devices such as drug delivery pumps, implanted drug infusion tubes, drug infusion catheters, and intravitreal drug delivery devices. Device 25 may also include, for example, ophthalmic devices such as scleral buckles and sponges, glaucoma drain shunts and intraocular lenses.

Device 25 may also include, for example, urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices. Device 25 may also include, for example, synthetic prostheses such as breast prostheses and artificial organs (e.g., pancreas, liver, lungs, heart, etc.). Device 25 may also include, for example, respiratory devices including lung catheters. Device 25 may also include, for example, neurological devices such as neurostimulators, neurological catheters, neurovascular balloon catheters, neuro-aneurysm treatment coils, and neuropatches, splints, ear wicks, ear drainage tubes, tympanostomy vent tubes, otological strips, laryngectomy tubes, esophageal tubes, esophageal stents, laryngeal stents, salivary bypass tubes, and tracheostomy tubes. Device 25 may also include, for example, oncological implants. Device 25 may also include, for example, pain management implants.

In some embodiments, device 25 is a non-implantable medical device, as discussed herein. Non-implantable devices can include dialysis devices and associated tubing, catheters, membranes, and grafts; autotransfusion devices; vascular and surgical devices including atherectomy catheters, angiographic catheters, intraaortic balloon pumps, intracardiac suction devices, blood pumps, blood oxygenator devices (including tubing and membranes), blood filters, blood temperature monitors, hemoperfusion units, plasmapheresis units, transition sheaths, dialators, intrauterine pressure devices, clot extraction catheters, percutaneous transluminal angioplasty catheters, electrophysiology catheters, breathing circuit connectors, stylets (vascular and non-vascular), coronary guide wires, peripheral guide wires; dialators (e.g., urinary, etc.); surgical instruments (e.g. scalpels and the like); endoscopic devices (such as endoscopic surgical tissue extractors, esophageal stethoscopes); and general medical and medically related devices including blood storage bags, umbilical tape, membranes, gloves, surgical drapes, wound dressings, wound management devices, needles, percutaneous closure devices, transducer protectors, pessary, uterine bleeding patches, PAP brushes, clamps (including bulldog clamps), cannulae, cell culture devices, materials for in vitro diagnostics, chromatographic support materials, infection control devices, colostomy bag attachment devices, birth control devices; disposable temperature probes; and pledgets.

Anchorage device 20 can have a variety of different configurations, shapes and sizes. For example, substrate 24 and/or substrate 28 can be provided with a size and shape or other configuration that can provide the functionality of supporting and immobilizing the medical device 25 at a treatment site within a patient's body, while also improving the removability of anchorage device 20 after the treatment has been completed. In some embodiments, medical device 25 can be disposed within cavity 34 and anchorage device 20 can be implanted and secured to tissue at a desired treatment site within a body of a patient. As discussed herein, during implantation, scar tissue can form at the treatment site and/or tissue can become ingrown within substrate 24 and/or substrate 28. After the treatment is completed, medical device 25 can remain in the patient as discussed below or can be removed from the patient leaving anchorage device 20 implanted. To remove anchorage device 20, tissue that is ingrown within substrate 24 and/or substrate 28 can be cut or otherwise detached from substrate 24 and/or substrate 28. In some embodiments, a portion of anchorage device 20 may not be removable from the tissue and will remain implanted within the patient.

Anchorage device 20 may be formed with one or more biocompatible materials, which may be synthetic or naturally occurring. In some embodiments, the one or more biocompatible materials include, for example, polypropylene, polyester, polytetrafluoroethylene, polyamides, silicones, polysulfones, metals, alloys, titanium, stainless steel, shape memory metals (e.g., Nitinol), and/or combinations thereof. In some embodiments, substrate 24 and/or substrate 28 is/are made at least in part from one or more hemostatic agents, such as, for example, collagen. In some embodiments, substrate 24 and/or substrate 28 is/are made entirely from a hemostatic agent, such as, for example, collagen. In some embodiments, substrate 24 and/or substrate 28 is/are free of any hemostatic agents such that any hemostatic agent of device 20 would be included in a coating that coats substrate 24 and/or substrate 28, rather from substrate 24 and/or substrate 28 itself.

In some embodiments, anchorage device 20 is configured to be implanted temporarily within a body of a patient and/or is configured to be removed (e.g., explanted) from the patient's body after a period of time. In such embodiments, substrate 24 and/or substrate 28 may include a non-biodegradable material and/or a non-bioresorbable material. For example, substrate 24 and/or substrate 28 may be made entirely from a non-biodegradable material and/or a non-bioresorbable material such that substrate 24 and/or substrate 28 is made only from the non-biodegradable material and/or non-bioresorbable material. In some embodiments, substrate 24 and/or substrate 28 may include one or more non-biodegradable and/or a non-bioresorbable material and one or more biodegradable and/or resorbable material. In some embodiments, one side of substrate 24 and/or substrate 28 may include one or more non-biodegradable and/or a non-bioresorbable material and another side of substrate 24 and/or substrate 28 can include one or more biodegradable and/or resorbable material.

As used herein, the term “biodegradable” refers to, for example, a material that can be at least partially broken down or degraded by a bodily fluid and discarded as waste from the body and/or a material that can be broken down or degraded by a living organism. Thus, “non-biodegradable” can refer to a material that cannot be broken down or degraded by a bodily fluid and/or cannot be broken down or degraded by a living organism. As used herein the term “resorbable” refers to, for example, a material that can be at least partially broken down or degraded by a bodily fluid and assimilated within the body. Thus, a “non-resorbable” material as used herein can refer to, for example, a material that cannot be broken down or degraded by bodily fluid and assimilated within the body.

In some embodiments, the biocompatible biodegradable and/or bioresorbable material or materials may include polymeric and/or non-polymeric materials, such as, for example, one or more poly (alpha-hydroxy acids), poly (lactide-co-glycolide) (PLGA), polylactide (PLA), poly(L-lactide), polyglycolide (PG), polyethylene glycol (PEG) conjugates of poly (alpha-hydroxy acids), polyorthoesters (POE), polyaspirins, polyphosphazenes, collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, POE, SAIB (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteram ides, tyrosine polyarylates, polyetheretherketone, polymethylmethacrylate, silicone, hyaluronic acid, chitosan, or combinations thereof. In one embodiment, substrate 24 and/or substrate 28 comprises Glycoprene, which is sold by Poly-Med, Inc. As used herein, the term “glycoprene” or “Glycoprene” refers to Glycoprene® or Glycoprene II®. Glycoprene® can refer to different variations of the material sold under the trade name Glycoprene®, such as, for example, Glycoprene® 6829, Glycoprene® 8609 and Glycoprene®7027.

In some embodiments, the biocompatible non-biodegradable and/or non-bioresorbable material or materials may include polymeric and/or non-polymeric materials, such as, for example, polyurethane, polyester, polytetrafluoroethylene (PTFE), polyethylacrylate/polymethylmethacrylate, polylactide, polylactide-co-glycolide, polyamides, polydioxanone, polyvinyl chloride, polymeric or silicone rubber, collagen, thermoplastics, or combinations thereof.

In some embodiments, anchorage device 20 is configured to be permanently implanted within a body of a patient. In such embodiments, substrate 24 and/or substrate 28 may include a biodegradable material and/or a bioresorbable material. For example, substrate 24 and/or substrate 28 may be made entirely from a biodegradable material and/or a bioresorbable material such that substrate 24 and/or substrate 28 is made only from the biodegradable material and/or bioresorbable material.

In some embodiments, substrate 24 and/or substrate 28 is provided in the form of a mesh. In some embodiments, the mesh is web or fabric with a construction of knitted, braided, woven or non-woven filaments or fibers that are interlocked in such a way to create a fabric or a fabric-like material that includes a matrix of filaments that define multiple pores. That is, the space between adjacent filaments or fibers define pores of the mesh. Pores may be beneficial to allow tissue in-growth, for example. In some embodiments, apertures may be formed in the mesh by cutting the filaments or fibers to decrease the areal density (e.g., surface density) or mass of the mesh and/or further facilitate tissue in-growth. In some embodiments, the apertures that extend through the filaments or fibers are larger than pores defined by the filaments or fibers.

In some embodiments, anchorage device 20 includes an overlay, such as, for example, a coating 38 that is applied to substrate 24 such that coating 38 covers all or a portion of substrate 24. In some embodiments, anchorage device 20 includes an overlay, such as, for example, a coating 40 that is applied to substrate 28 such that coating 40 covers all or a portion of substrate 28.

In some embodiments, coating 38 includes collagen, glycerol and tranexamic acid (TXA) and is configured to be applied directly to substrate 24. In some embodiments, coating 38 comprises between about 0.1 wt. % and about 10 wt. % collagen, between about 90 wt. % and about 99 wt. % water, between about 0.1 wt. % and about 3.0 wt. %) glycerol, between about 0.1 wt. % and about 5.0 wt. % TXA and between about 0.1 wt. % and about 8.0 wt. % 1N NaOH. In some embodiments, coating 38 comprises between about 1.2 wt. % and about 5.4 wt. % collagen, between about 87.1 wt. % and about 97.1 wt. % water, between about 0.2 wt. % and about 2.2 wt. % glycerol, between about 0.1 wt. % and about 2.0 wt. % TXA and between about 0.3 wt. % and about 4.3 wt. % 1N NaOH. In some embodiments, coating 38 comprises about 3.4 wt. % collagen, about 92.1 wt. % water, about 1.2 wt. % glycerol, about 0.9 wt. % TXA and about 2.3 wt. % 1N NaOH. In some embodiments, coating 38 comprises 3.4 wt. % collagen, 92.1 wt. % water, 1.2 wt. % glycerol, 0.9 wt. % TXA and 2.3 wt. % 1N NaOH. In some embodiments, water may be reduced by up to a factor of 10. In some embodiments, coating 38 includes ellagic acid in place of or in addition to the other components of coating 38 discussed herein.

In some embodiments, coating 38 can include one or more hemostatic agent (HA) and coating 40 can include one or more active pharmaceutical ingredient (API). In some embodiments, coating 38 is free of any polymer such that the HA is applied directly to substrate 24 in the form of a powder, for example. In some embodiments, coating 40 is free of any polymer such that the API is applied directly to substrate 28 in the form of a powder, for example.

In some embodiments, the HA and the API are each dispersed within a polymer, such as, for example, one or more of the polymers discussed herein such that the polymer degrades to release the HA and the API upon implantation of device 20. For example, coating 38 can include a first polymer that includes the HA dispersed therein such that the first polymer releases the HA as the first polymer degrades and coating 40 can include a second polymer that includes the API dispersed therein such that the second polymer releases the API as the second polymer degrades. In some embodiments, the first and second polymers are the same polymer. In some embodiments, the first and second polymers are different polymers.

In some embodiments, substrate 24 is biodegradable and/or bioresorbable and device 20 is configured to hold medical device 25 and/or substrate 28 therein such that substrate 24 does not begin to degrade until the first polymer of coating 38 completely degrades such that device 20 can hold medical device 25 and/or substrate 28 therein until all of the HA is released from the first polymer of coating 38. In some embodiments, substrate 24 is completely biodegradable or bioresorbable. That all of substrate 24 is biodegradable or bioresorbable. In some embodiments, substrate 24 is completely non-biodegradable and/or non-bioresorbable. That is no portion of substrate 24 is biodegradable or bioresorbable. In some embodiments, substrate 24 and/or coating 38 are free of any APIs, such as, for example, the APIs discussed herein.

In some embodiments, substrate 28 is biodegradable and/or bioresorbable and device 20 is configured to hold medical device 25 therein such that substrate 28 does not begin to degrade until the second polymer of coating 40 completely degrades such that device 20 can hold medical device 25 therein until all of the API is released from the second polymer of coating 40. In some embodiments, substrate 28 is completely biodegradable or bioresorbable. That all of substrate 28 is biodegradable or bioresorbable. In some embodiments, substrate 28 is completely non-biodegradable and/or non-bioresorbable. That is no portion of substrate 28 is biodegradable or bioresorbable. In some embodiments, substrate 28 and/or coating 40 are free of any HAs, such as, for example, the HAs discussed herein.

The HA can include one or more hemostatic agents, such as, for example, epinephrine, tranexamic acid, collagen, chitosan and oxidized regenerated cellulose. In some embodiments, the collagen can include acid soluble collagen, pepsin soluble collagen, gelatin, cross-linkable collagen, fibrillar collagen. In some embodiments, the HA can include one or more of Spongostan®, Surgifoam®, Avitene, thrombin and Ostene® in addition to or in place of the hemostatic agents discussed above. In some embodiments, the HA can include one or more of protamine, norepinephrine, desmopressin, lysine analogs, gelatin, polysaccharide spheres, mineral zeolite, bovine thrombin, pooled human thrombin, recombinant thrombin, gelatin and thrombin, collagen and thrombin, cyanacrylate, fibrin glue, polyethylene glycol, and glutaraldehyde in addition to or in place of the hemostatic agents discussed above. In some embodiments, the HA includes a mixture or combination of the HAs discussed herein. In some embodiments, the lysine analog is tranexamic acid.

In some embodiments, the anchorage devices disclosed herein utilize one or more pharmacologic hemostatic agent since pharmacologic hemostatic agents have been found to be desirable over mechanical hemostats for a variety of reasons. Ethnographic research has showed that physicians desire a hemostat that can provide an extended elution profile to reduce bleeding events for up to 7 days post operatively. Furthermore, there is a possible effect on handling and/or allergic reactions if mechanical hemostats, such as, for example, oxidized reduced cellulose or chitosan were used.

In some embodiments, tranexamic acid is preferred for use as the HA. Tranexamic acid is a synthetic analog of the amino acid lysine with a molecular weight of 157 g/mol. Tranexamic acid is an antifibrinolytic agent that acts by binding to plasminogen and blocking the interaction of plasminogen with fibrin, therefore preventing the dissolution of a fibrin clot. In the presence of a wound, fibrinolysis occurs naturally when a lysine residue such as tissue plasminogen activator (tPA), binds to plasmin causing the clot to lyse (or break). Tranexamic acid blocks tPA and keeps the clot from breaking, thus preventing unwanted bleeding.

Prior to a damaged endothelium, tPA is inhibited in the blood by plasminogen activator inhibitor/type 1 (PAI-1). Once damage occurs, the tPA is released slowly into the blood, activating fibrinolysis. Excessive fibrinolysis results in a condition called hvperfibrinolysis, which requires intervention such as fibrinogen, plasma, transfusion or antifibrinolytic therapy, such as tranexamic acid.

Tranexamic acid has been used for over 40 years to reduce bleeding complications. Tranexamic acid is most commonly given systemically at doses of 10 mg/kg followed by infusion of 10 mg/kg/h. Since 2007, tranexamic acid has received widespread approval and clinical use as a hemostatic agent. Knowing that surgical trauma causes fibrinolysis in the area of the surgical wound itself, topical antifibrinolytic therapy is becoming more common to obtain and maintain hemostasis. Clinical trials with topical tranexamic acid use exist for cardiac surgery, CIED procedures, orthopedic surgery, spinal surgery, dental extraction and epistaxis, and breast mammoplasty.

To evaluate the efficacy of tranexamic acid, a non-GLP acute porcine study was conducted. Doses of 1 mg to 200 mg of tranexamic acid were used in an in vitro whole blood coagulation test, a hepatic biopsy test, and a subcutaneous ICD surgical procedure.

The in vitro whole blood coagulation test showed no activity for tranexamic acid up to 10 mg/ml. The maximum tranexamic acid concentration, 200 mg/5 ml, was a slightly higher dose than that used clinically in a CIED pocket if 50cc is the assumed blood volume of interest. Coagulation time was doubled with this higher dose.

The hepatic biopsy test had a volume of 0.016 ml when the biopsy hole was filled with blood. The minimum tranexamic acid dose evaluated was 2.5 mg, which is equivalent to 156 mg/ml. This concentration prevents blood from clotting quickly and these biopsies continued to bleed past the endpoint of 10 minutes. This phenomenon is likely due to the multiple bonding sites available to tranexamic acid in whole blood, and the fact that a biopsy does not induce fibrinolysis.

The subcutaneous surgical site test was conducted with an elevated ACT using heparin to induce hematoma. Surgical trauma similar to that of a CIEO implant was incurred in each pocket, but some subcutaneous pockets incurred more trauma than others due to anatomical location. The primary output monitored was accumulated blood as measured by pre-weighed gauze 3-hours post-operatively. With only one animal, and two pockets per treatment, the sample size was too low to show any significance between ICD only, ICD+polymer, and ICD+polymer+tranexamic acid.

The non-GLP acute porcine study showed that in the dose range evaluated, tranexamic acid has a two-fold increase on clotting time and no effect on reducing bleeding on the hepatic biopsies. In the heparinized ICD pocket procedure, 3.5-22.8 grams of blood accumulated in a 3-hour period of time regardless of treatment. It appears that subcutaneous pockets in an anticoagulated porcine model would be a translatable model for evaluating efficacy of tranexamic acid because it has a relevant volume of accumulated blood and surgical trauma similar to that of a CIED procedure.

Based upon the non-GLP acute porcine study, tranexamic acid concentrations of 3.00 mg/L to 30 mg/L are effective in preventing fibrinolysis. As such, in some embodiments, the HA is tranexamic acid and is provided in concentrations of about 3.00 mg/L to about 30 mg/L. However, it has been found that one tenth of the doses used in the non-GLP acute porcine study can be effective in reversing fibrinolysis. As such, in some embodiments, the HA is tranexamic acid and is provided in concentrations of about 0.30 mg/L to about 3.0 mg/L for intravenous applications. In some embodiments, tranexamic acid is provided in concentrations of about 3.78 mg/L to about 30 mg/L for topical applications as well. However, in some embodiments, however, higher doses of tranexamic acid are used for topical applications to account for tranexamic acid being widely distributed throughout the extracellular and intracellular compartments when given preoperatively. Indeed, it has been found that tranexamic acid reaches plasma concentrations in 5-15 minutes. As such, in some embodiments, tranexamic acid is provided in doses of about 1.5 mg to about 150 mg.

In some embodiments, substrate 24 is formed at least in part from hemostatic agent HA, as discussed herein. That is, substrate 24 is a hemostatic substrate that is made from hemostatic agent HA. In some embodiments, hemostatic substrate 24 is made only from hemostatic agent HA. In some embodiments, hemostatic agent HA does not include any coating, such as, for example, coating 38. In some embodiments, hemostatic agent HA includes a coating, such as, for example, coating 38. In some embodiments, coating 38 that is applied to hemostatic substrate 24 may include any of the coatings discussed herein.

Coatings 38, 40 applied to substrates 24, 28 such that anchorage device 20 delivers hemostatic agent HA in combination with the API. The API can include one or a combination of active pharmaceutical ingredients, such as, for example, anesthetics, antibiotics, anti-inflammatory agents, procoagulant agents, fibrosis-inhibiting agents, anti-scarring agents, antiseptics, leukotriene inhibitors/antagonists, cell growth inhibitors and mixtures thereof. In some embodiments, the API is an antibiotic. In some embodiments, the antibiotic is selected from the group consisting of rifampin and minocycline and mixtures thereof.

Examples of non-steroidal anti-inflammatories include, but are not limited to, naproxen, ketoprofen, ibuprofen as well as diclofenac; celecoxib; sulindac; diflunisal; piroxicam; indomethacin; etodolac; meloxicam; r-flurbiprofen; mefenamic; nabumetone; tolmetin, and sodium salts of each of the foregoing; ketorolac bromethamine; ketorolac bromethamine tromethamine; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, 1, and racemic isomers); and the methyl, ethyl, propyl, isopropyl, n-butyl, sec-butyl, t-butyl, esters of acetylsalicylic acid.

Examples of anesthetics include, but are not limited to, licodaine, bupivacaine, and mepivacaine. Further examples of analgesics, anesthetics and narcotics include, but are not limited to acetaminophen, clonidine, benzodiazepine, the benzodiazepine antagonist flumazenil, lidocaine, tramadol, carbamazepine, meperidine, zaleplon, trimipramine maleate, buprenorphine, nalbuphine, pentazocain, fentanyl, propoxyphene, hydromorphone, methadone, morphine, levorphanol, and hydrocodone. Local anesthetics have weak antibacterial properties and can play a dual role in the prevention of acute pain and infection.

Examples of antibacterial agents or antimicrobials include, but are not limited to, triclosan, chlorohexidine and other cationic biguanides, rifampin, minocycline (or other tetracycline derivatives), vancomycin, gentamycin; gendine; genlenol; genfoctol; clofoctol; cephalosporins and the like. Further antibacterial agents or antimicrobials include aztreonam; cefotetan and its disodium salt; loracarbef; cefoxitin and its sodium salt; cefazolin and its sodium salt; cefaclor; ceftibuten and its sodium salt; ceftizoxime; ceftizoxime sodium salt; cefoperazone and its sodium salt; cefuroxime and its sodium salt; cefuroxime axetil; cefprozil; ceftazidime; cefotaxime and its sodium salt; cefadroxil; ceftazidime and its sodium salt; cephalexin; hexachlorophene; cefamandole nafate; cefepime and its hydrochloride, sulfate, and phosphate salt; cefdinir and its sodium salt; ceftriaxone and its sodium salt; cefixime and its sodium salt; cetylpyridinium chloride; ofoxacin; linexolid; temafloxacin; fleroxacin; enoxacin; gemifloxacin; lomefloxacin; astreonam; tosufloxacin; clinafloxacin; cefpodoxime proxetil; chloroxylenol; methylene chloride, iodine and iodophores (povidone-iodine); nitrofurazone; meropenem and its sodium salt; imipenem and its sodium salt; cilastatin and its sodium salt; azithromycin; clarithromycin; dirithromycin; erythromycin and hydrochloride, sulfate, or phosphate salts ethylsuccinate, and stearate forms thereof, clindamycin; clindamycin hydrochloride, sulfate, or phosphate salt; lincomycin and hydrochloride, sulfate, or phosphate salt thereof, tobramycin and its hydrochloride, sulfate, or phosphate salt; streptomycin and its hydrochloride, sulfate, or phosphate salt; vancomycin and its hydrochloride, sulfate, or phosphate salt; neomycin and its hydrochloride, sulfate, or phosphate salt; acetyl sulfisoxazole; colistimethate and its sodium salt; quinupristin; dalfopristin; amoxicillin; ampicillin and its sodium salt; clavulanic acid and its sodium or potassium salt; penicillin G; penicillin G benzathine, or procaine salt; penicillin G sodium or potassium salt; carbenicillin and its disodium or indanyl disodium salt; piperacillin and its sodium salt; a-terpineol; thymol; taurinamides; nitrofurantoin; silver-sulfadiazine; hexetidine; methenamine; aldehydes; azylic acid; silver; benzyl peroxide; alcohols; carboxylic acids; salts; nafcillin; ticarcillin and its disodium salt; sulbactam and its sodium salt; methylisothiazolone, moxifloxacin; amifloxacin; pefloxacin; nystatin; carbepenems; lipoic acids and its derivatives; beta-lactams antibiotics; monobactams; aminoglycosides; microlides; lincosamides; glycopeptides; tetracyclines; chloramphenicol; quinolones; fucidines; sulfonamides; macrolides; ciprofloxacin; ofloxacin; levofloxacins; teicoplanin; mupirocin; norfloxacin; sparfloxacin; ketolides; polyenes; azoles; penicillins; echinocandines; nalidixic acid; rifamycins; oxalines; streptogramins; lipopeptides; gatifloxacin; trovafloxacin mesylate; alatrofloxacin mesylate; trimethoprims; sulfamethoxazole; demeclocycline and its hydrochloride, sulfate, or phosphate salt; doxycycline and its hydrochloride, sulfate, or phosphate salt; minocycline and its hydrochloride, sulfate, or phosphate salt; tetracycline and its hydrochloride, sulfate, or phosphate salt; oxytetracycline and its hydrochloride, sulfate, or phosphate salt; chlortetracycline and its hydrochloride, sulfate, or phosphate salt; metronidazole; dapsone; atovaquone; rifabutin; linezolide; polymyxin B and its hydrochloride, sulfate, or phosphate salt; sulfacetamide and its sodium salt; and clarithromycin (and combinations thereof). In some embodiments the polymer may contain rifampin and another antimicrobial agent, such as, for example, an antimicrobial agent that is a tetracycline derivative. In some embodiments, the polymer contains a cephalosporin and another antimicrobial agent. In some embodiments, the polymer contains combinations including rifampin and minocycline, rifampin and gentamycin, and rifampin and minocycline.

When a mixture of two antibiotics is used, they generally present in a ratio ranging from about 10:1 to about 1:10. In some embodiments, a mixture of rifampin and minocycline are used. In those embodiments, a ratio of rifampin to minocycline ranges from about 5:2 to about 2:5. In other embodiments, the ratio of rifampin to minocycline is about 1:1.

Examples of antifungals include amphotericin B; pyrimethamine; flucytosine; caspofungin acetate; fluconazole; griseofulvin; terbinafine and its hydrochloride, sulfate, or phosphate salt; amorolfine; triazoles (Voriconazole); flutrimazole; cilofungin; LY303366 (echinocandines); pneumocandin; imidazoles; omoconazole; terconazole; fluconazole; amphotericin B, nystatin, natamycin, liposomal amptericin B, liposomal nystatins; griseofulvin; BF-796; MTCH 24; BTG-137586; RMP-7/Amphotericin B; pradimicins; benanomicin; ambisome; ABLC; ABCD; Nikkomycin Z; flucytosine; SCH 56592; ER30346; UK 9746; UK 9751; T 8581; LY121019; ketoconazole; micronazole; clotrimazole; econazole; ciclopirox; naftifine; and itraconazole.

In some embodiments, active pharmaceutical ingredient API includes keflex, acyclovir, cephradine, malphalen, procaine, ephedrine, adriamycin, daunomycin, plumbagin, atropine, quinine, digoxin, quinidine, biologically active peptides, cephradine, cephalothin, cis-hydroxy-L-proline, melphalan, penicillin V, aspirin, nicotinic acid, chemodeoxycholic acid, chlorambucil, paclitaxel, sirolimus, cyclosporins, 5-fluorouracil and the like.

In some embodiments, the API includes one or more ingredients that act as angiogenensis inhibitors or inhibit cell growth such as epidermal growth factor, PDGF, VEGF, FGF (fibroblast growth factor) and the like. These ingredients include anti-growth factor antibodies (neutrophilin-1), growth factor receptor-specific inhibitors such as endostatin and thalidomide. Examples of useful proteins include cell growth inhibitors such as epidermal growth factor.

Examples of anti-inflammatory compounds include, but are not limited to, anecortive acetate; tetrahydrocortisol, 4,9(11)-pregnadien-17α, 21-diol-3,20-dione and its -21-acetate salt; 111-epicortisol; 17α-hydroxyprogesterone; tetrahydrocortexolone; cortisona; cortisone acetate; hydrocortisone; hydrocortisone acetate; fludrocortisone; fludrocortisone acetate; fludrocortisone phosphate; prednisone; prednisolone; prednisolone sodium phosphate; methylprednisolone; methylprednisolone acetate; methylprednisolone, sodium succinate; triamcinolone; triamcinolone-16,21-diacetate; triamcinolone acetonide and its -21-acetate, -21-disodium phosphate, and -21-hemisuccinate forms; triamcinolone benetonide; triamcinolone hexacetonide; fluocinolone and fluocinolone acetate; dexamethasone and its -21-acetate, -21-(3,3-dimethylbutyrate), -21-phosphate disodium salt, -21-diethylaminoacetate, -21-isonicotinate, -21-dipropionate, and -21-palmitate forms; betamethasone and its -21-acetate, -21-adamantoate, -17-benzoate, -17,21-dipropionate, -17-valerate, and -21-phosphate disodium salts; beclomethasone; beclomethasone dipropionate; diflorasone; diflorasone diacetate; mometasone furoate; and acetazolamide.

Examples of leukotriene inhibitors/antagonists include, but are not limited to, leukotriene receptor antagonists such as acitazanolast, iralukast, montelukast, pranlukast, verlukast, zafirlukast, and zileuton.

In some embodiments, active pharmaceutical ingredient API includes sodium 2-mercaptoethane sulfonate (“MESNA”). MESNA has been shown to diminish myofibroblast formation in animal studies of capsular contracture with breast implants [Ajmal et al. (2003) Plast. Reconstr. Surg. 112:1455-1461] and may thus act as an anti-fibrosis agent.

Procoagulants include, but are not limited to, zeolites, thrombin, and coagulation factor concentrates.

In some embodiments, the amount of the API that is applied to hemostatic substrate 28 via coating 40 or otherwise ranges between about 0.3 to about 2.8 micrograms/cm². In other embodiments, the amount of the API that is applied to substrate 28 via coating 40 or otherwise ranges between about 0.6 to about 1.4 micrograms/cm². In yet other embodiments, the amount of the API that is applied to substrate 28 via coating 40 or otherwise ranges between about 0.85 to about 1.20 micrograms/cm². In yet further embodiments, the amount of the API that is applied to substrate 28 via coating 40 or otherwise ranges between about 0.90 to about 1.10 micrograms/cm². In yet further embodiments, the amount of the API that is applied to substrate 28 via coating 40 or otherwise ranges between about 50 to about 150 micrograms/cm². In yet further embodiments, the amount of the API that is applied to substrate 28 via coating 40 or otherwise ranges between about 62 to about 140 micrograms/cm². In yet further embodiments, 62 micrograms/cm² of the API is applied to substrate 28 via coating 40 or otherwise. In yet further embodiments, 140 micrograms/cm² of the API is applied to substrate 28 via coating 40 or otherwise. In some embodiments, a first amount of the API is applied to substrate 28 via coating 40 and a second amount is applied to substrate 28 via a powder that is applied to substrate 28 after coating 40 is applied to substrate 28. For example, anchorage device 20 may be delivered to a medical practitioner with coating 40 being pre-applied to substrate 28 and including a standard amount of the API. The medical practitioner may then apply a powder, gel, slurry, solution, etc. of the API to the pre-applied coating 40 to add an additional amount of the API to anchorage device 20.

In some embodiments, the amount of the HA that is applied to substrate 24 via coating 38 or otherwise ranges between about 0.3 to about 2.8 micrograms/cm². In other embodiments, the amount of the HA that is applied to substrate 24 via coating 38 or otherwise ranges between about 0.6 to about 1.4 micrograms/cm². In yet other embodiments, the amount of the HA that is applied to substrate 24 via coating 38 or otherwise ranges between about 0.85 to about 1.20 micrograms/cm². In yet further embodiments, the amount of the HA that is applied to substrate 24 via coating 38 or otherwise ranges between about 0.90 to about 1.10 micrograms/cm². In yet further embodiments, the amount of the HA that is applied to substrate 24 via coating 38 or otherwise ranges between about 50 to about 150 micrograms/cm². In yet further embodiments, the amount of the HA that is applied to substrate 24 via coating 38 or otherwise ranges between about 62 to about 140 micrograms/cm². In yet further embodiments, 62 micrograms/cm² of the HA is applied to substrate 24 via coating 38 or otherwise. In yet further embodiments, 140 micrograms/cm² of the HA is applied to substrate 24 via coating 38 or otherwise. In some embodiments, a first amount of the HA is applied to substrate 24 via coating 38 and a second amount of the HA is applied to substrate 24 via a powder that is applied to substrate 24 after coating 38 is applied to substrate 24. For example, anchorage device 20 may be delivered to a medical practitioner with coating 38 being pre-applied to substrate 24 and including a standard amount of the HA. The medical practitioner may then apply a powder, gel, slurry, solution, etc. of the HA to the pre-applied coating 38 to add an additional amount of the HA to anchorage device 20.

In some embodiments, the amount of the HA and the API that is applied to substrates 24, 28 via coatings 38, 40 or otherwise ranges between about 0.3 to about 2.8 micrograms/cm². In other embodiments, the amount of the HA and the API that is applied to substrates 24, 28 via coatings 38, 40 or otherwise ranges between about 0.6 to about 1.4 micrograms/cm². In yet other embodiments, the amount of the HA and the API that is applied to substrates 24, 28 via coatings 38, 40 or otherwise ranges between about 0.85 to about 1.20 micrograms/cm². In yet further embodiments, the amount of the HA and the API that is applied to substrates 24, 28 via coatings 38, 40 or otherwise ranges between about 0.90 to about 1.10 micrograms/cm². In yet further embodiments, the amount of the HA and the API that is applied to substrates 24, 28 via coatings 38, 40 or otherwise ranges between about 50 to about 150 micrograms/cm². In yet further embodiments, the amount of the HA and the API that is applied to substrates 24, 28 via coatings 38, 40 or otherwise ranges between about 62 to about 140 micrograms/cm². In yet further embodiments, 62 micrograms/cm² of the HA and the API is applied to substrates 24, 28 via coatings 38, 40 or otherwise. In yet further embodiments, 140 micrograms/cm² of the HA and the API is applied to substrates 24, 28 via coatings 38, 40 or otherwise. In some embodiments, a first amount of the HA and the API is applied to substrates 24, 28 via coatings 38, 40 and a second amount of the HA and the API is applied to substrates 24, 28 via a powder that is applied to substrates 24, 28 after coatings 38, 40 are applied to substrate 22. For example, anchorage device 20 may be delivered to a medical practitioner with coatings 38, 40 being pre-applied to substrates 24, 28 and including a standard amount of the HA and the API. The medical practitioner may then apply a powder, gel, slurry, solution, etc. of the HA and the API to the pre-applied coatings 38, 40 to add an additional amount of the HA and the API to anchorage device 20.

In other embodiments, the API includes rifampin and minocycline and the amount of each of rifampin and minocycline that is applied to substrate 28 via coating 40 or otherwise ranges between about 0.6 to about 1.4 micrograms/cm². In yet other embodiments, the amount of each of rifampin and minocycline that is applied to substrate 28 via coating 40 or otherwise ranges between about 0.85 to about 1.20 micrograms/cm². In yet further embodiments, the amount of each of rifampin and minocycline that is applied to substrate 28 via coating 40 or otherwise ranges between about 0.90 to about 1.10 micrograms/cm². In some embodiments, a first amount of the rifampin and minocycline is applied to substrate 28 via coating 40 or otherwise and a second amount of the rifampin and minocycline is applied to substrate 28 via a powder that is applied to substrate 28 after coating 40 is applied to substrate 28. For example, anchorage device 20 may be delivered to a medical practitioner with coating 40 being pre-applied to substrate 28 and including a standard amount of the rifampin and minocycline. The medical practitioner may then apply a powder, gel, slurry, solution, etc. of the rifampin and minocycline to the pre-applied coating 40 to add an additional amount of the rifampin and minocycline to anchorage device 20.

The API may include one or more of the active pharmaceutical ingredients discussed herein. The API may be incorporated into anchorage device 20 by applying the API directly to substrate 28 or by applying the API to substrate 28 via a polymer, such as, for example, one or more of the polymers discussed herein. Doses of the APIs discussed herein are known and the amounts of any single API to include in anchorage device 20 can readily be surmised. Any pharmaceutically acceptable form of APIs discussed herein can be employed in anchorage device 20, e.g., the free base or a pharmaceutically acceptable salt or ester thereof. Pharmaceutically acceptable salts, for instance, include sulfate, lactate, acetate, stearate, hydrochloride, tartrate, maleate, citrate, phosphate and the like.

The polymer discussed herein, such as, for example, the polymer of coating 38 that contains the HA and/or the polymer of coating 40 that includes the API is selected from the group consisting of polylactic acid, polyglycolic acid, poly(L-lactide), poly(D,L-lactide)polyglycolic acid[polyglycolide], poly(L-lactide-co-D,L-lactide), poly(L-lactide-co-glycolide), poly(D, L-lactide-co-glycolide), poly(glycolide-co-trimethylene carbonate), poly(D,L-lactide-co-caprolactone), poly(glycolide-co-caprolactone), polyethylene oxide, polydioxanone, polypropylene fumarate, poly(ethyl glutamate-co-glutamic acid), poly(tert-butyloxy-carbonylmethyl glutamate), polycaprolactone, polycaprolactone co-butylacrylate, polyhydroxybutyrate, copolymers of polyhydroxybutyrate, poly(phosphazene), poly(phosphate ester), poly(amino acid), polydepsipeptides, maleic anhydride copolymers, polyiminocarbonates, poly[(97.5% dimethyl-trimethylene carbonate)-co-(2.5% trimethylene carbonate)], poly(orthoesters), tyrosine-derived polyarylates, tyrosine-derived polycarbonates, tyrosine-derived polyiminocarbonates, tyrosine-derived polyphosphonates, polyethylene oxide, polyethylene glycol, polyalkylene oxides, hydroxypropylmethylcellulose, polysaccharides such as hyaluronic acid, chitosan and regenerate cellulose. In some embodiments, the polymer of coating 38 that contains the HA and/or the polymer of coating 40 that includes the API may include combinations, blends or mixtures of the polymers discussed herein.

In some embodiments, the polymer of coating 38 that contains the HA and/or the polymer of coating 40 that includes the API is a polyarylate. In some embodiments, the polymer of coating 38 that contains the HA and/or the polymer of coating 40 that includes the API is a tyrosine-derived polyarylate. In some embodiments, the tyrosine-derived polyarylate is p(DTE co X % DT succinate), where X is about 10% to about 30%. In some embodiments, the tyrosine-derived polyarylate is p(DTE co X % DT succinate), where X ranges from about 26.5% to about 28.5%. In some embodiments, the tyrosine-derived polyarylate is p(DTE co X % DT succinate), where X is about 27.5%. In some embodiments, the polymer of coating 38 that contains the HA and/or the polymer of coating 40 that includes the API is P22-27.5 DT.

As used herein, DTE is the diphenol monomer desaminotyrosyl-tyrosine ethyl ester; DTBn is the diphenol monomer desaminotyrosyl-tyrosine benzyl ester; DT is the corresponding free acid form, namely desaminotyrosyl-tyrosine. BTE is the diphenol monomer 4-hydroxy benzoic acid-tyrosyl ethyl ester; BT is the corresponding free acid form, namely 4-hydroxy benzoic acid-tyrosine.

P22-XX is a polyarylate copolymer produced by condensation of DTE and DTBn with succinic acid followed by removal of benzyl group. P22-10, P22-15, P22-20, P22-XX, etc., represents copolymers different percentage of DT (i.e., 10, 15, 20 and % DT, etc.) In some embodiments, the polymer is produced by condensation of DTBn with succinic acid followed by removal of benzyl group. This polymer is represented as P02-100.

In some embodiments, the polymer of coating 38 that contains the HA and/or the polymer of coating 40 that includes the API includes one or more polyarylates that are copolymers of desaminotyrosyl-tyrosine (DT) and an desaminotyrosyl-tyrosyl ester (DT ester), wherein the copolymer comprises from about 0.001% DT to about 80% DT and the ester moiety can be a branched or unbranched alkyl, alkylaryl, or alkylene ether group having up to 18 carbon atoms, any group of which can, optionally have a polyalkylene oxide therein. Similarly, another group of polyarylates are the same as the foregoing but the desaminotyrosyl moiety is replaced by a 4-hydroxybenzoyl moiety. In some embodiments, the DT or BT contents include those copolymers with from about 1% to about 30%, from about 5% to about 30% from about 10 to about 30% DT or BT. In some embodiments, the diacids (used informing the polyarylates) include succinate, glutarate and glycolic acid.

In some embodiments, the polymer of coating 38 that contains the HA and/or the polymer of coating 40 that includes the API includes one or more biodegradable, resorbable polyarylates and polycarbonates. These polymers, include, but are not limited to, BTE glutarate, DTM glutarate, DT propylamide glutarate, DT glycineamide glutarate, BTE succinate, BTM succinate, BTE succinate PEG, BTM succinate PEG, DTM succinate PEG, DTM succinate, DT N-hydroxysuccinimide succinate, DT glucosamine succinate, DT glucosamine glutarate, DT PEG ester succinate, DT PEG amide succinate, DT PEG ester glutarate, DT PEG ester succinate, DTMB P(Desaminotyrsoyl tyrosine methylparaben ester-glutarate), and DTPP P(Desaminotyrsoyl tyrosine propylparaben ester-glutarate).

In some embodiments, the polymer of coating 38 that contains the HA and/or the polymer of coating 40 that includes the API is one more polymers from the DTE-DT succinate family of polymers, e.g., the P22-xx family of polymers having from 0-50%, 5-50%, 5-40%, 1-30% or 10-30% DT, including but not limited to, about 1, 2, 5, 10, 15, 20, 25, 27.5, 30, 35, 40%, 45% and 50% DT. In some embodiments, the polymer is P22-27.5 DT.

In some embodiments, the polymer of coating 38 that contains the HA and/or the polymer of coating 40 that includes the API has diphenol monomer units that are copolymerized with an appropriate chemical moiety to form a polyarylate, a polycarbonate, a polyiminocarbonate, a polyphosphonate or any other polymer.

In some embodiments, the polymer of coating 38 that contains the HA and/or the polymer of coating 40 that includes the API is tyrosine-based polyarylate. In some embodiments, the polymer of coating 38 that contains the HA and/or the polymer of coating 40 that includes the API includes blends and copolymers with polyalkylene oxides, including polyethylene glycol (PEG).

In some embodiments, the polymer of coating 38 that contains the HA and/or the polymer of coating 40 that includes the API can have from 0.1-99.9% PEG diacid to promote the degradation process. In some embodiments, the polymer of coating 38 that contains the HA and/or the polymer of coating 40 that includes the API includes blends of polyarylates or other biodegradable polymers with polyarylates.

The polymer of coating 38 that contains the HA and/or the polymer of coating 40 that includes the API is configured to release the HA and/or the API over time, as discussed herein. In some embodiments, the polymer of coating 38 that contains the HA and/or the polymer of coating 40 that includes the API is configured to release the HA and/or the API over a time period ranging from about 1 hour to about 168 hours. In some embodiments, the polymer of coating 38 that contains the HA and/or the polymer of coating 40 that includes the API is configured to release the HA and/or the API over a time period ranging from 1 hour to 72 hours. In some embodiments, the polymer of coating 38 that contains the HA and/or the polymer of coating 40 that includes the API is configured to release the HA and/or the API over a time period ranging from 1 hour to 24 hours.

In some embodiments, the polymer of coating 38 that contains the HA and/or the polymer of coating 40 that includes the API is configured to release the HA and/or the API over time in an area surrounding or adjacent to anchorage device 20 (such as, for example, within the device “pocket” or within 3 inches in all dimensions). In some embodiments, the polymer of coating 38 that contains the HA and/or the polymer of coating 40 that includes the API is configured to release the HA and/or the API for up to 30 hours. In some embodiments, the polymer of coating 38 that contains the HA and/or the polymer of coating 40 that includes the API is configured to release between about 40% and about 100% of the HA and/or the API over a period of at least about 30 hours. In some embodiments, the polymer of coating 38 that contains the HA and/or the polymer of coating 40 that includes the API is configured to release 60% and about 100% of the HA and/or the API over a period of at least about 30 hours. In some embodiments, the polymer of coating 38 that contains the HA and/or the polymer of coating 40 that includes the API is configured to release between about 65% and about 100% of the HA and/or the API over a period of at least about 36 hours. In some embodiments, the polymer of coating 38 that contains the HA and/or the polymer of coating 40 that includes the API is configured to release 80% and about 100% of the HA and/or the API over a period of at least about 36 hours. In some embodiments, the polymer of coating 38 that contains the HA and/or the polymer of coating 40 that includes the API is configured to release between about 60% and about 100% of the HA and/or the API over a period of at least about 48 hours. In some embodiments, the polymer of coating 38 that contains the HA and/or the polymer of coating 40 that includes the API is configured to release 80% and about 100% of the HA and/or the API over a period of at least about 48 hours. In some embodiments, the polymer of coating 38 that contains the HA and/or the polymer of coating 40 that includes the API is configured to release between about 60% and about 100% of the HA and/or the API over a period of at least about 60 hours. In some embodiments, the polymer of coating 38 that contains the HA and/or the polymer of coating 40 that includes the API is configured to release 80% and about 100% of the HA and/or the API over a period of at least about 60 hours. In some embodiments, the polymer of coating 38 that contains the HA and/or the polymer of coating 40 that includes the API is configured to release 80% and about 100% of the HA and/or the API within 48 hours. In some embodiments, the polymer of coating 38 that contains the HA and/or the polymer of coating 40 that includes the API is configured to release 80% and about 100% of the HA and/or the API within 24 hours.

In some embodiments, the polymer of coating 38 that contains the HA and/or the polymer of coating 40 that includes the API is configured to release no more than 60% of the HA and/or the API within 24 hours. In some embodiments, the polymer of coating 38 that contains the HA and/or the polymer of coating 40 that includes the API is configured to release no more than 90% of the HA and/or the API after 60 hours. In some embodiments, the polymer of coating 38 that contains the HA and/or the polymer of coating 40 that includes the API is configured to release no more than 50% of the HA and/or the API within 12 hours. In some embodiments, the polymer of coating 38 that contains the HA and/or the polymer of coating 40 that includes the API is configured to release between about 40% and about 90% of the HA and/or the API between 12 and 24 hours. In some embodiments, the polymer of coating 38 that contains the HA and/or the polymer of coating 40 that includes the API is configured to release between about 60% and about 100% of the HA and/or the API between 24 and 36 hours. In some embodiments, the polymer of coating 38 that contains the HA and/or the polymer of coating 40 that includes the API is configured to release between about 65% and about 100% of the HA and/or the API between 36 and 48 hours. In some embodiments, the polymer of coating 38 that contains the HA and/or the polymer of coating 40 that includes the API is configured to release between about 70% and about 100% of the HA and/or the API between 48 and 60 hours.

Substrate 24 may be coated with single or multiple coating layers of coating 38, depending on, for example, the amount of the HA to be delivered and desired release rate. Each layer of coating 38 may contain the same or different amounts of the HA. For example, a first layer of coating 38 may contain the HA, while the second layer of coating 38 contains either no HA or a lower concentration of the HA. As another example, a first layer of coating 38 may comprise the HA in a first polymer, while the second layer of coating 38 comprises the HA in a second polymer that is different than the first polymer.

Substrate 28 may be coated with single or multiple coating layers of coating 40, depending on, for example, the amount of the API to be delivered and desired release rate. Each layer of coating 40 may contain the same or different amounts of the API. For example, a first layer of coating 40 may contain the API, while the second layer of coating 40 contains either no API or a lower concentration of the API. As another example, a first layer of coating 40 may comprise the API in a first polymer, while the second layer of coating 40 comprises the API in a second polymer that is different than the first polymer.

In embodiments discussed herein wherein component 22 is a pocket or envelope, a first coating 38 can be applied to first piece 24a and a second coating 38 can be applied to second piece 24b. In some embodiments, the first and second coatings 38 are different. In some embodiments, the first and second coatings 38 release the HA at different rates and/or over different lengths of time. In some embodiments, the first coating 38 includes a first amount of the HA and the second coating 38 includes a second amount of the HA, the first amount being different than the second amount. In some embodiments, the first and second coatings 38 are the same. In some embodiments, the first and second coatings 38 include different HAs.

In embodiments discussed herein wherein component 26 is a pocket or envelope, a first coating 40 can be applied to first piece 28a and a second coating 40 can be applied to second piece 28b. In some embodiments, the first and second coatings 40 are different. In some embodiments, the first and second coatings 40 release the API at different rates and/or over different lengths of time. In some embodiments, the first coating 40 includes a first amount of the API and the second coating 40 includes a second amount of the API, the first amount being different than the second amount. In some embodiments, the first and second coatings 40 are the same. In some embodiments, the first and second coatings 40 include different APIs.

In some embodiments, anchorage device 20 includes a hydrophilic component, such as, for example, PEG and a crosslinking agent that is applied to substrate 24 and/or substrate 28. The hydrophilic component and the crosslinking agent form a hydrogel that absorbs blood and reduces bleeding when in contact with blood or tissue fluid. In some embodiments, the hydrophilic component and the crosslinking agent are sprayed directly onto substrate 24 and/or substrate 28. In some embodiments, the hydrophilic component and the crosslinking agent are provided in a polymer, such as, for example, one or more of the polymers discussed herein, and the polymer is applied directly onto substrate 24 and/or substrate 28. In some embodiments, the hydrophilic component and the crosslinking agent are provided in a patch, such as, for example, the Veriset™ hemostatic patch available from Medtronic, Inc., and the patch is applied directly onto substrate 24 and/or substrate 28.

In some embodiments, the hydrophilic component comprises thermogelling hydrogels, PEG-PLGA copolymers, PEG-Poly(N-isopropyl acrylamide), Pluronic (PEO-PPO-PEO triblock), PEG-PCL polymers, PEG-based amphiphilic copolymers modified by anionic weak polyelectrolytes, (such as polyacrylic acid, polyglutamic acid) and polymers containing sulfonamide groups), PEG-based amphiphilic copolymers modified by cationic weak polyelectrolytes (such as poly (2-vinyl pyridine), Poly(beta-amino esters), poly (2-(dimethylamino)ethyl methacrylate), multiarm PEG derivatives such as those available from JenKem technology, multiarmed block and graft PLA copolymers with PEG, PEG with stereo complexed poly(lactide), acrylated polymers (such as Polyvinylalcohol, dextran, Polyvinylpyrollidone, chitosan, alginate, hyaluronic acid), and combinations thereof. In some embodiments, the crosslinking agent comprises one or more agents that induce polymerization of vinyl groups using various initiators, light or redox reactions, or by reactions such as Schiff base formation, Michael type additions, peptide ligation, clock chemistry of functional groups present; one or more agents that induce crosslinking by enzymatic reaction (transglutaminase mediated reaction between carboxamide and amine on proteins), stereo-complexation, metal chelation (alginates using calciumCal2), thermogelation, self-assembly (formation of super helices from protein chains) inclusion complexation (using cyclodextrin); and combinations thereof.

In some embodiments, an anchorage device, such as, for example, anchorage device 20 and a medical device, such as, for example, medical device 25 are implanted into a body of a patient. The anchorage device releases a hemostatic agent and an active pharmaceutical ingredient, such as, for example, the HA and/or the API, to reduce or prevent bleeding within the patient or treat one of the conditions as discussed herein. In some embodiments, anchorage device 20 is implanted within the patient without medical device 25 and medical device 25 is coupled to or inserted into cavity 34 after anchorage device 20 is implanted. In some embodiments, medical device 25 is coupled to or inserted into cavity 34 before anchorage device 20 is implanted within the patient and anchorage device 20 and medical device 25 are implanted within the patient together.

In some embodiments, medical device 25 is removed from the patient after the treatment is completed. In some embodiments, anchorage device 20 remains implanted within the patient after medical device 25 is removed. In some embodiments, anchorage device 20 is removed from the patient after medical device 25 is removed. To remove anchorage device 20, tissue that is ingrown within substrate 22 of anchorage device 22 can be cut or otherwise detached from substrate 22. In some embodiments, a portion of anchorage device 20 may not be removable from the tissue and will remain implanted within the patient.

In some embodiments, component 26 is configured to be removably positioned in cavity 34. That is, component 26 is configured to inserted into cavity 34 and then be removed from cavity 34 at a later time. In such embodiments, component 26 is not joined with component 22 in order to allow component 26 to be removed from cavity 34. That is, anchorage device 20 does not include any structural components or substances that join or bond component 26 with component 22 in a manner that would prevent component 26 from being removed from cavity 34.

In some embodiments, component 26 is coupled to and/or joined with component 22 in a manner that prevents component 26 from being removed from cavity 34. That is, component 26 is coupled to and/or joined with component 22 in a manner that prevents component 26 from being removed from cavity 34 without damaging and/or destroying component 22 and/or component 26.

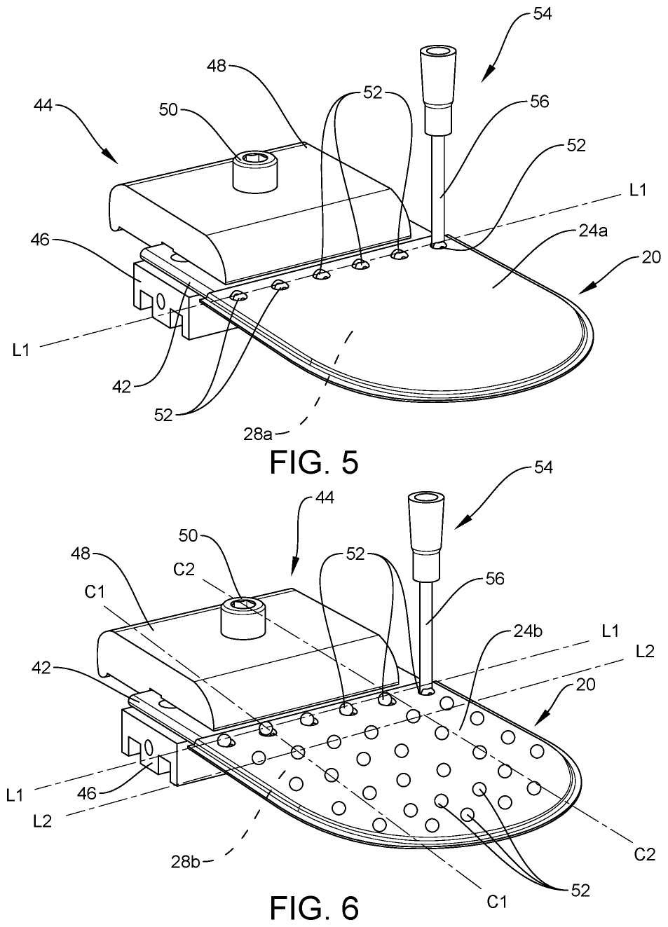

In one embodiment, shown in FIG. 5, component 26 is coupled to and/or joined with component 22 in a manner that prevents component 26 from being removed from cavity 34 by staking. In particular, after second component 26 is inserted into cavity 30, as shown in FIG. 2, for example, a plate 42 is inserted into cavity 34, as shown in FIG. 5. Plate 42 and anchorage device 20 are coupled to a mount 44 by positioning an end of plate 42 on a base 46 of mount 44 and positioning a clamp 48 of mount 44 over plate 42 such that plate 42 is positioned between base 46 and clamp 48. An actuator, such as, for example, a screw 50 extends through clamp 48 and into base 46 such that rotation of screw in a first rotational direction, such as, for example, clockwise relative to base 46 and clamp 48 moves clamp 48 toward base 46. Screw 50 is rotated in the first rotational direction until base 46 and clamp 48 engage opposite sides of plate 42 to prevent movement of plate 42 relative to base 46 and clamp 48. As shown in FIG. 5, base 46 and clamp 48 do not directly engage any portion of anchorage device 20 to prevent damage to anchorage device 20 caused by moving clamp 48 toward base 46, as discussed herein. However, in some embodiments, plate 42 and anchorage device 20 may be positioned relative to mount 44 such that base 46 directly engages one of first piece 24a and second piece 24b while clamp 48 directly engages the other one first piece 24a and second piece 24b to prevent movement of anchorage device 20 relative to plate 42.

Once plate 42 is fixed relative to base 46 and clamp 48, one or more stakes 52 are inserted through first piece 24a or second piece 24b of substrate 24 and into first piece 28a or second piece 28b of substrate 28. As shown in FIG. 5, plate 42 and anchorage device 20 are positioned relative to mount 44 such that first pieces 24a, 28a face away from base 46. When plate 42 and anchorage device 20 are positioned relative to mount 44 such that first pieces 24a, 28a face away from base 46, stakes 52 are inserted through first piece 24a of substrate 24 and into first piece 28a of substrate 28. However, in some embodiments, plate 42 and anchorage device 20 are positioned relative to mount 44 such that second pieces 24b, 28b face away from base 46 to allow stakes 52 to be inserted through second piece 24b of substrate 24 and into second piece 28b of substrate 28, as discussed herein.

In some embodiments, plate 42 and anchorage device 20 are positioned relative to mount 44 such that first pieces 24a, 28a face away from base 46. Once plate 42 is fixed relative to mount 44 as discussed herein, stakes 52 are inserted through first piece 24a of substrate 24 and into first piece 28a of substrate 28. Once stakes 52 are inserted through first piece 24a of substrate 24 and into first piece 28a of substrate 28, plate 42 and anchorage device 20 are uncoupled from mount 44 by rotating screw 50 in an opposite second rotational direction, such as, for example, counterclockwise. Plate 42 is then moved away from mount and rotated 180 degrees. After plate 42 is rotated 180 degrees, plate 42 is positioned on base 46 such that second pieces 24b, 28b face away from base 46 and screw 50 is inserted through clamp 48 and into base 46 and is rotated in the first rotational direction relative to base 46 and clamp 48 until plate 42 is fixed relative to base 46 and clamp 48. Stakes 52 are then inserted through second piece 24b of substrate 24 and into second piece 28b of substrate 28. That is, component 26 is joined with component 22 either by inserting stakes 52 are inserted through first piece 24a of substrate 24 and into first piece 28a of substrate 28 and inserting stakes 52 through second piece 24b of substrate 24 and into second piece 28b of substrate 28. However, in some embodiments, component 26 is joined with component 22 either by inserting stakes 52 are inserted through first piece 24a of substrate 24 and into first piece 28a of substrate 28 or by inserting stakes 52 through second piece 24b of substrate 24 and into second piece 28b of substrate 28.

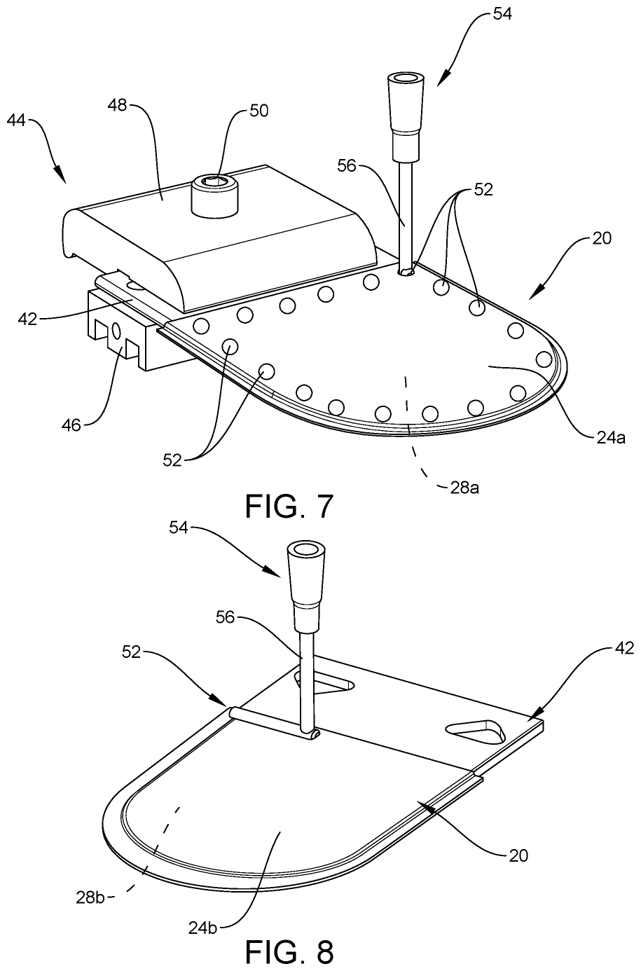

In some embodiments, system 15 includes a robotically controlled dispensing system that applies stakes 52 to anchorage device 20 to join component 26 with component 22 in a manner that prevents removal of component 26 from cavity 30, as shown in FIG. 5. In some embodiments, stakes 52 are dispensed through a tip 56 of robotically controlled dispensing system 54. In particular, robotically controlled dispensing system 54 includes a part, such as, for example, an arm that is configured to selectively move tip 56 relative to anchorage device 20 to insert stakes 52 into selected portions of anchorage device 20. For example, in some embodiments, robotically controlled dispensing system 54 is adapted to provide a plurality of spaced apart stakes 52 that are arranged in a straight line L1, as shown in FIG. 5. However, in other embodiments, controlled dispensing system 54 is adapted to provide a plurality of spaced apart stakes 52 that are arranged in a plurality of spaced apart straight lines, such as, for example, spaced apart straight lines L1, L2, wherein lines L1, L2 extend parallel to one another, as shown in FIG. 6. In some embodiments, anchorage device 20 may include one or a plurality of straight lines of spaced apart stakes 52 in addition to lines L1, L2, wherein the additional lines extend parallel to lines L1, L2 and are spaced apart from lines L1, L2. In some embodiments, robotically controlled dispensing system 54 is adapted to provide a plurality of spaced apart stakes 52 such that stakes 52 in one or more of lines L1, L2 and/or the additional lines that extend parallel to lines L1, L2 are arranged in a column defined by a straight line that extends perpendicular to lines L1, L2 or the additional lines that extend parallel to lines L1, L2. For example, robotically controlled dispensing system 54 is adapted to define a plurality of columns of spaced apart stakes 52, such as, for example, spaced apart columns C1, C2 of stakes 52 shown in FIG. 6. It is envisioned that the number of rows of stakes 52, such as, for example, lines L1, L2 and/or the number of columns of stakes 52, such as, for example, columns C1, C2 can be varied based on the requirements of a particular application. In some embodiments, robotically controlled dispensing system 54 is adapted to insert stakes 52 into anchorage device 20 such that stakes 52 are uniformly spaced apart from one another. In some embodiments, robotically controlled dispensing system 54 is adapted to insert stakes 52 into anchorage device 20 such that stakes 52 are arranged randomly. In some embodiments, robotically controlled dispensing system 54 is adapted to insert stakes 52 into anchorage device 20 such that stakes 52 extend about all or a portion of a perimeter of anchorage device, as shown in FIG. 7, for example.

In some embodiments, robotically controlled dispensing system 54 is adapted to insert one or a plurality of stakes 52 into anchorage device 20 such that stake(s) 52 extends in a continuous line across at least a portion of anchorage device, as shown in FIG. 8, for example. In some embodiments wherein stake(s) 52 is/are in a continuous line, it is envisioned that anchorage device 20 can include spaced apart continuously lines of stakes 52. In some embodiments wherein stake(s) 52 is/are in a continuous line, it is envisioned that anchorage device 20 can include spaced apart continuously lines of stakes 52 that are arranged in rows similar to lines L1, L2 and/or columns similar to columns C1, C2 or in any other selected pattern, depending upon the requirements of a particular application.

In some embodiments, stakes 52 include collagen, such as, for example, gelling collagen. In some embodiments, stakes 52 include a collagen rich solution In some embodiments, at least one of stakes 52 includes a first amount of collagen. In some embodiments, at least one of stakes 52 includes an amount of collagen that is significantly more than the first amount of collagen. In some embodiments, at least one of stakes 52 includes an amount of collagen up to 50% more than the first amount of collagen. In some embodiments, stakes 52 include an ultraviolet (UV) curable solution. In some embodiments, robotically controlled dispensing system 54 deposits stakes 52 onto anchorage device 20 wherein stakes 52 are in the form of droplets that move through first piece 24a or second piece 24b and into first piece 28a or second piece 28.

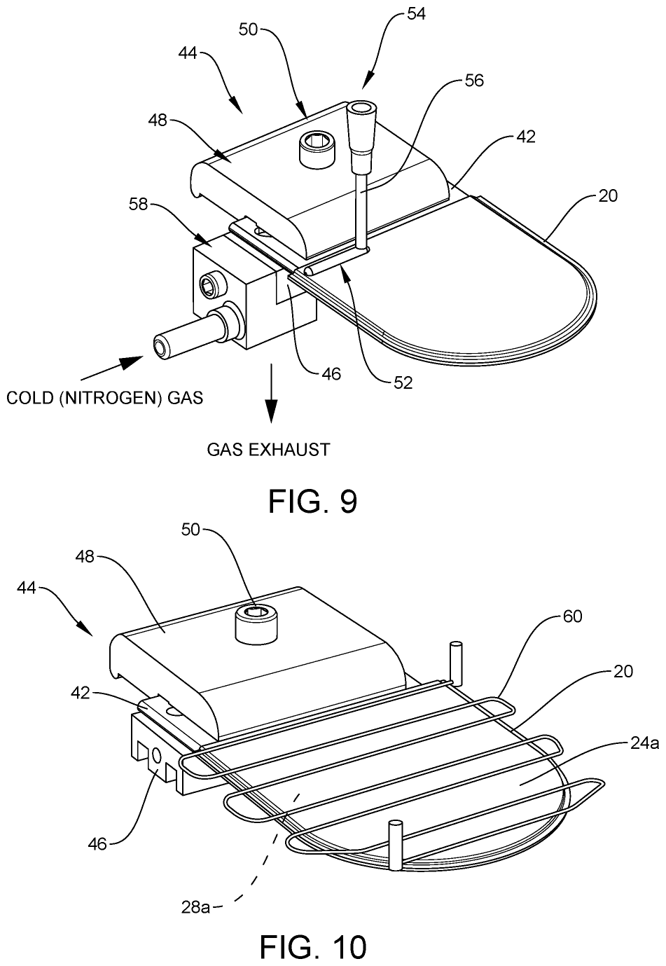

In some embodiments, UV light is applied to anchorage device 20 after stakes 52 are inserted into anchorage device 20 to promote curing of stakes 52. In some embodiments, anchorage device 20 is cooled after stakes 52 are inserted into anchorage device 20 to promote curing of stakes 52. In some embodiments, anchorage device 20 is cooled via plate 42. In particular, in some embodiments, mount 44 includes and/or is coupled to a cooling block assembly 58 (FIG. 9) wherein cooling block assembly 58 is configured to cool plate 42 via a refrigerated system to a temperature between −10 degrees Celsius and −20 degrees Celsius. In some embodiments, cooling block assembly 58 is configured to cool plate 42 via a refrigerated system to −15 degrees Celsius. In some embodiments, the refrigerated system uses liquid nitrogen boil-off gas that is applied directly to plate 42 and anchorage device 20. In some embodiments, cold gas (nitrogen, dry air) is blown over stakes 52 as a cover gas.

In some embodiments, anchorage device 20 is trimmed after stakes 52 are inserted to join component 26 with component 22 in a manner that prevents component 26 from being removed from cavity 34. For example, in some embodiments, excess material, such as, for example, the material that forms substrate 24 and/or coating 38 is trimmed from component 22 after stakes 52 are inserted to join component 26 with component 22. In some embodiments, excess material is material that it is not needed for the function of anchorage device 20 such as material that overhangs the perimeter of anchorage device 22. In some embodiments the material to be joined (staked) to 28b is unrolled, staked (via stakes 52 as discussed herein and an excess material is then trimmed after staking. In some embodiments, excess material, such as, for example, the material that forms substrate 28 and/or coating 40 is trimmed from component 26 after stakes 52 are inserted to join component 26 with component 22. In some embodiments, excess material is removed from component 22 and/or component 26 via laser trimming. In some embodiments, excess material is removed from component 22 and/or component 26 via die punch trimming.

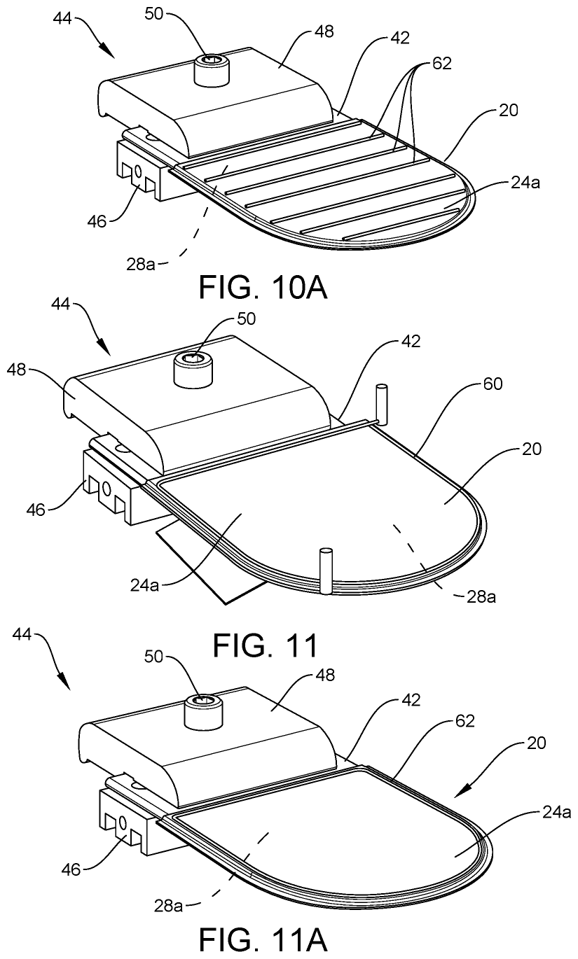

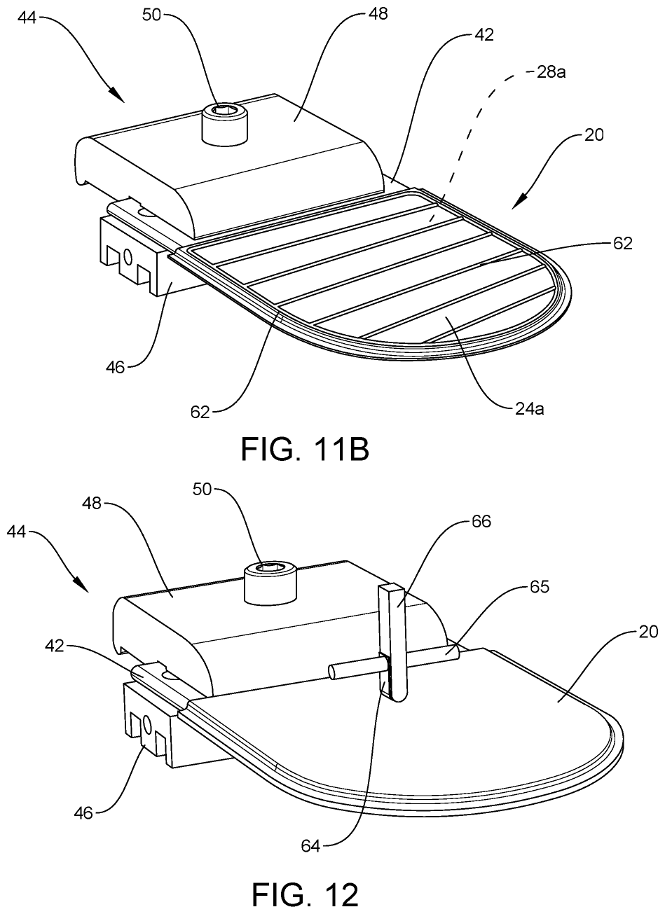

In some embodiments, component 26 is coupled to and/or joined with component 22 in a manner that prevents component 26 from being removed from cavity 34 by one or more heat seal bands 60 of system 15, as shown in FIGS. 10 and 11. In particular, after component 26 is inserted into cavity 30, plate 42 is inserted into cavity 34 and is fixed relative to base 46 and clamp 48 via screw 50 in the manner discussed above. Heat seal band 60 is then pressed into an outer surface of first piece 24a or an outer surface of second piece 24b, as shown in FIGS. 10 and 11, to create one or a plurality of seals 62 in anchorage device 20. That is, heat seal band 60 is pressed into anchorage device 20 until heat from heat seal band 60 moves through component 22 and into component 26 to create seals 62, which join component 26 with component 22 in a manner that prevents component 26 from being removed from cavity 34. In some embodiments, heat seal band 60 is connected to two cylindrical poles, shown in FIG. 10, for example, that define electrical contacts and conductors to heat seal band 60. In some embodiments, the conductors (cylindrical poles) are big enough (low DC resistance) so as not to increase in heat, while heat seal band 60 increases in heat. Seal(s) 62 is/are visible after heat seal band 60 is removed from anchorage device, as shown in FIGS. 10A and 11A. In one embodiment, shown in FIGS. 10 and 10A, the configuration of heat seal band 60 creates a plurality of spaced apart seals 62. In some embodiments, seals 62 extend horizontally across anchorage device 20 and are spaced apart from one another. In some embodiments, at least one of seals 62 extends across an entire width of anchorage device 20. In one embodiment, shown in FIGS. 11 and 11A, the configuration of heat seal band 60 creates a single seal 62. In some embodiments, seal 62 extends about all or a portion of a perimeter of anchorage device 20. In that that there is a direct relationship between the shape of heat seal band 60 and the configuration of seal(s) 62 on anchorage device 20, it is envisioned that heat seal band 60 can be configured to have any shape that would result in a selected configuration of seal(s) 62 on anchorage device 20. For example, all or a portion of heat seal band 60 can be variously shaped, such as, for example, circular, oval, oblong, triangular, square, rectangular, elliptical, polygonal, irregular, uniform, non-uniform, offset, staggered, undulating, arcuate, variable and/or tapered.

In some embodiments, component 26 is coupled to and/or joined with component 22 in a manner that prevents component 26 from being removed from cavity 34 by at least two heat seal bands 60. In particular, after component 26 is inserted into cavity 30, plate 42 is inserted into cavity 34 and is fixed relative to base 46 and clamp 48 via screw 50 in the manner discussed above. In some embodiments, a first heat seal band 60 that is the same or similar to heat seal band 60 in FIG. 10 can be used in the manner discussed above to create seals 62 in anchorage device 20 that are the same or similar to that shown in FIG. 10A. Once seals 62 in anchorage device 20 that are the same or similar to that shown in FIG. 10A are created, a second heat seal band 60 that is the same or similar to heat seal band 60 in FIG. 11 can be used in the manner discussed above to create seal 62 in anchorage device 20 that is the same or similar to that shown in FIG. 11A. As a result, anchorage device 20 will have seals 62 shown in FIG. 10A and seal 62 shown in FIG. 11A, as shown in FIG. 11B.

In some embodiments, seal(s) 62 may be created between pieces 24a, 28a and/or between pieces 24b, 28b in the manner discussed above. For example, heat seal band 60 may be first used to create seal(s) 62 between pieces 24a, 28a by applying heat seal band 60 to pieces 24a, 28a. Once seal(s) 62 are created between pieces 24a, 28a, plate 42 can be uncoupled from mount 44 in the manner discussed above and repositioned relative to mount 44 such that pieces 24b, 28b face away from base 46. Heat seal band 60 may then be used to create seal(s) 62 between pieces 24b, 28b by applying heat seal band 60 to pieces 24b, 28b.

In some embodiments, an interface is positioned between anchorage device 20 and heat seal band 60 to facilitate the release of heat seal band 60 from anchorage device 20 after seal(s) 62 is/are formed into anchorage device 20 by heat seal band 60. In some embodiments, the interface includes Kapton tape. In some embodiments, the Kapton tape is positioned between heat seal band 60 and a surface of anchorage device 20 (i.e., first piece 24a or second piece 24b). The tape holds heat seal band 60 in a selected position on anchorage device 20 and allows for reliable release of heat seal band 60 from anchorage device 20. In some embodiments, another material configured for heat tolerance may be used in addition to or in place of the Kapton tape.

In some embodiments, anchorage device 20 is trimmed after seal(s) 62 is/are created by one or more heat seal bands 60 to join component 26 with component 22 in a manner that prevents component 26 from being removed from cavity 34. For example, in some embodiments, excess material, such as, for example, the material that forms substrate 24 and/or coating 38 is trimmed from component 22 after seal(s) 62 is/are created by one or more heat seal bands 60 to join component 26 with component 22. In some embodiments, excess material, such as, for example, the material that forms substrate 28 and/or coating 40 is trimmed from component 26 after seal(s) 62 is/are created by one or more heat seal bands 60 to join component 26 with component 22. In some embodiments, excess material is removed from component 22 and/or component 26 via laser trimming. In some embodiments, excess material is removed from component 22 and/or component 26 via die punch trimming.

In some embodiments, component 26 is coupled to and/or joined with component 22 in a manner that prevents component 26 from being removed from cavity 34 by a heat seal band 64 of system 15, wherein heat seal band 64 is coupled to a post 66 that is movably controlled by a robot arm, for example, that is the same or similar to the robot arm of robot controlled dispensing system 54 discussed above, as shown in FIG. 12. In particular, after component 26 is inserted into cavity 30, plate 42 is inserted into cavity 34 and is fixed relative to base 46 and clamp 48 via screw 50 in the manner discussed above. Once plate 42 and anchorage device 20 are fixed relative to base 46 and clamp 48, a component of system 15, such as, for example, a robot arm is configured to be coupled to post 66 and move post 66 in a plurality of different directions relative to plate 42 and anchorage device 20 to allow heat seal band 64 to selectively create one or more seals that are the same or similar to seals 62 between component 22 and component 26 to join component 26 with component 22. In some embodiments, system 15 includes one or more conductors 65. In some embodiments, conductor(s) 65 extend(s) perpendicular to post 66. In some embodiments, conductor(s) 65 may be disposed at alternate orientations, relative to post 66, such as, for example, transverse and/or other angular orientations such as acute or obtuse, co-axial and/or may be offset or staggered.

In some embodiments, heat seal band 64 is configured to create spaced apart seals. In some embodiments, heat seal band 64 is configured to one or more seals that extend across at least a portion of anchorage device 20. In some embodiments, heat seal band 64 is configured to create seals that are arranged in a pattern. In some embodiments, heat seal band 64 is configured to create one or more seals that form a continuous line. In some embodiments, heat seal band 64 is configured to create one or more seals that extend about at least a portion of a perimeter of anchorage device 20.

In some embodiments, an interface is positioned between anchorage device 20 and heat seal band 64 to facilitate the release of heat seal band 64 from anchorage device 20 after the seal(s) is/are formed into anchorage device 20 by heat seal band 64. In some embodiments, the interface includes Kapton tape.

In some embodiments, anchorage device 20 is trimmed after the seal(s) is/are created by heat seal band 64 to join component 26 with component 22 in a manner that prevents component 26 from being removed from cavity 34 in a manner that is the same or similar to the in which anchorage device 20 is trimmed after seal(s) 62 is/are created by one or more heat seal bands 60 to join component 26 with component 22 in a manner that prevents component 26 from being removed from cavity 34.

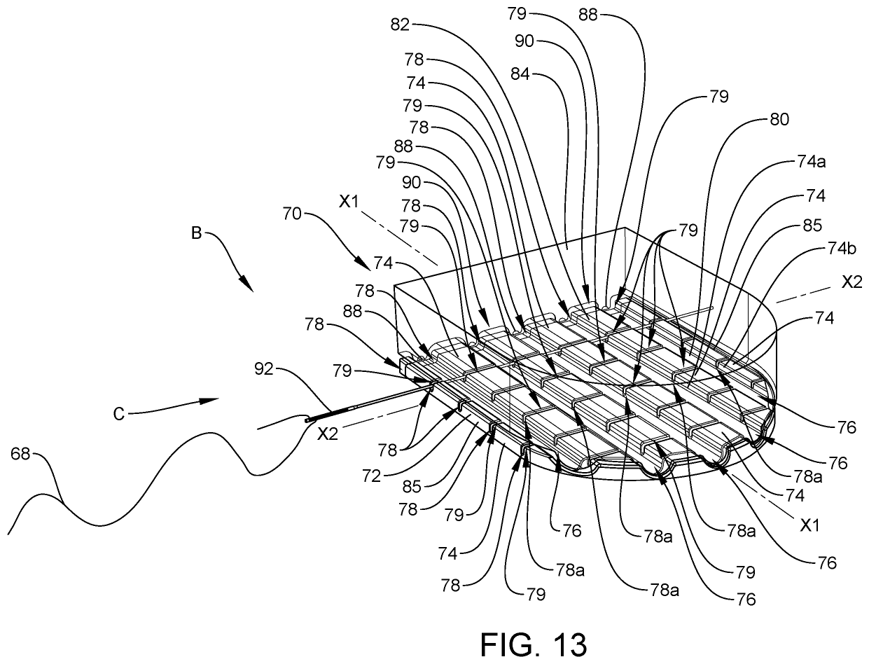

In some embodiments, component 26 is coupled to and/or joined with component 22 in a manner that prevents component 26 from being removed from cavity 34 by inserting one or more sutures 68 into anchorage device 20 using a press 70, as shown in FIGS. 13-15. Press 70 includes a lower mandrel, such as, for example, a base 72 having a plurality of spaced apart rails 74 that define channels 76 therebetween. In some embodiments, rails 74 and channels 76 provide base 72 with a corrugated configuration. Rails 74 extend parallel to one another along a longitudinal axis X1, as shown in FIG. 13. Base 72 includes a plurality of spaced apart holes 78 that each extend through opposite sides 74a, 74b of one of rails 74 or a side wall 85 of base 82. Holes 78 extend along a transverse axis X2 that extends perpendicular to longitudinal axis X1. Holes 78 are arranged into a plurality of spaced apart series of holes 78 wherein each series of holes 78 includes holes 78 that extend through each of rails 74 or one of side walls 85 and are coaxial with one another along transverse axis X2. Illustrated in FIG. 13 is an example of a series of holes 78, which is shown by holes 78a in FIG. 13. Holes 78 are each positioned in one of rails 74 between a bottom wall 80 of base 72 and a top surface 82 of one of rails 74. In some embodiments, holes 78 are each positioned in one of rails 74 equidistant between bottom wall 80 of base 72 and top surface 82 of one of rails 74. In some embodiments, transverse axis X2 may be disposed at alternate orientations, relative to longitudinal axis X1, such as, for example, transverse and/or other angular orientations such as acute or obtuse and/or may be offset or staggered.

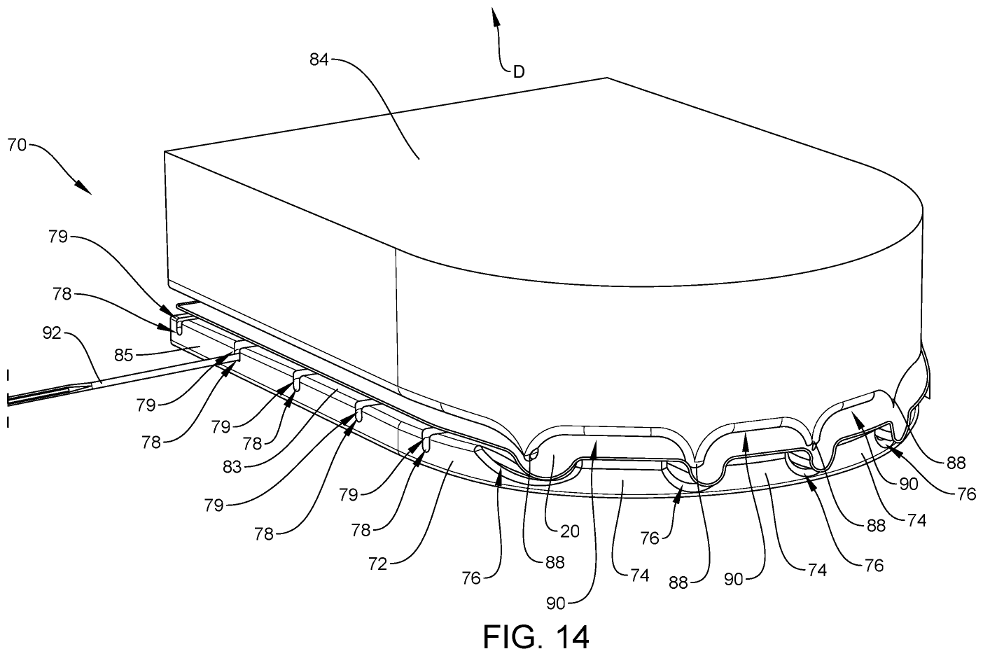

In some embodiments, base 72 further includes a plurality of passageways 79 that each extend through surface 80 or proximal surface 83 of base 82 and are each in communication with one of holes 78, as shown in FIG. 14, for example. In some embodiments, passageways 79 extend perpendicular to axis X1 and/or axis X2. In some embodiments, passageways 79 each have a uniform diameter, such as, for example, a uniform maximum diameter. In some embodiments, the maximum diameter of passageways 79, such as, for example, the maximum uniform diameters of passageways 79 each extends continuously from one of holes 78 to surface 83. In some embodiments, passageways 79 each have a maximum diameter that is equal to or substantially equal to the maximum diameters of holes 72. In some embodiments, passageways 79 each have a maximum diameter that is less than the maximum diameters of holes 72. In some embodiments, passageways 79 each have a maximum diameter that is greater than the maximum diameters of holes 72. In some embodiments, passageways 79 may be disposed at alternate orientations, relative to axis X1 and/or axis X2, such as, for example, transverse, perpendicular and/or other angular orientations such as acute or obtuse, co-axial and/or may be offset or staggered.

To insert one or more sutures 68 into anchorage device 20, component 26 is inserted into cavity 30 in the manner discussed above. Anchorage device 20 is positioned over base 72 with anchorage device 20 having a planar or substantially planar configuration that is the same or similar to the configuration of anchorage device 20 shown in FIGS. 2-4. Anchorage device 20 is then pressed into base 72 such that first portions of anchorage device 20 are inserted into channels 76 such that the first portions of anchorage device 20 contact bottom wall 80 of base 72. Second portions of anchorage device 20 extend across top surfaces 82 of rails 74 when the first portions of anchorage device 20 are inserted into channels 76.

In some embodiments, anchorage device 20 is pressed into base 72 by positioning anchorage device 20 over base 72 with anchorage device in the planar or substantially planar configuration discussed above and then positioning a press foot, such as, for example, a plate 84 of press 70 over anchorage device 20 such that anchorage device is positioned between base 72 and plate 84. Plate 84 includes a wall 86 and a plurality of spaced apart ribs 88 extending from wall 86. Adjacent ribs 88 define a groove 90 therebetween.

In some embodiments, plate 84 includes a plurality of grooves 89 each extending through each of ribs 88, as best shown in FIG. 16. Grooves 89 are positioned such that grooves 89 are aligned with holes 78 and/or channels 79 when plate 84 is coupled to base 72. That is, plate 84 includes a plurality of series of grooves 89 that each extend through ribs 88 and are coaxial with one another along transverse axis X2 when plate 84 is coupled to base 72. As such, when plate 84 is coupled to base 72, one of the series of grooves 89 that each extend through ribs 88 and are coaxial with one another along transverse axis X2 when plate 84 is coupled to base 72 will be coaxial with one of the series of holes 78 that extend through each of rails 74 and are coaxial with one another along transverse axis X2 to allow the series of grooves 89 and the series of holes 78 to define a pathway for a suture and/or needle that extends through an entire width of base 72 and/or an entire width of plate 84. In some embodiments, the series of grooves 89 is coaxial with the series of holes 78 to define the pathway for the suture and/or needle.

Anchorage device 20 is pressed into base 72 by moving plate 84 relative to base 72 in the direction shown by arrow B in FIG. 13 such that each of ribs 88 moves into one of channels 76, each of rails 74 moves into one of grooves 90 and wall 86 is positioned over top surfaces 82 of rails 74. As ribs 88 each move into one of channels 76 and rails 74 each moves into one of grooves 90 the first portions of anchorage device 20 move into channels 76 to move anchorage device from the planar or substantially planar configuration to a corrugated configuration that corresponds to the corrugated configuration of base 72, as shown in FIG. 18.

One of sutures 68 is coupled to a needle 92 and needle 92 is inserted into a first hole 78 of one of the series of holes 78 that extend through each of rails 74 and are coaxial with one another along transverse axis X2 and one of the series of grooves 89 that each extend through ribs 88 and are coaxial with one another along transverse axis X2 in a first direction along transverse axis X2, such as, for example, the direction shown by arrow C in FIG. 13. Needle 92 is further inserted into base 72 in the direction shown by arrow C such that needle 92 extends through each of holes 78 in the respective one of the series of holes 78 that extend through each of rails 74 and are coaxial with one another along transverse axis X2 and each of grooves 89 in the respective one of the series of grooves 89 that each extend through ribs 88 and are coaxial with one another along transverse axis X2. Needle 92 is then moved in the direction shown by arrow C in FIG. 13 such that needle 92 is spaced apart and/or removed from the respective one of the series of holes 78 that extend through each of rails 74 and are coaxial with one another and the respective one of the series of grooves 89 that each extend through ribs 88 and are coaxial with one another along transverse axis X2, leaving suture 68 extending through the respective one of the series of holes 78 that extend through each of rails 74 and are coaxial with one another and components 22, 26 of anchorage device 20 and the respective one of the series of grooves 89 that each extend through ribs 88 and are coaxial with one another along transverse axis X2 to join component 26 with component 22 in a manner that prevents component 26 from being removed from cavity 30. Suture 68 will be positioned in holes 78 and groove 89 after suture 68 is threaded through anchorage device 20.

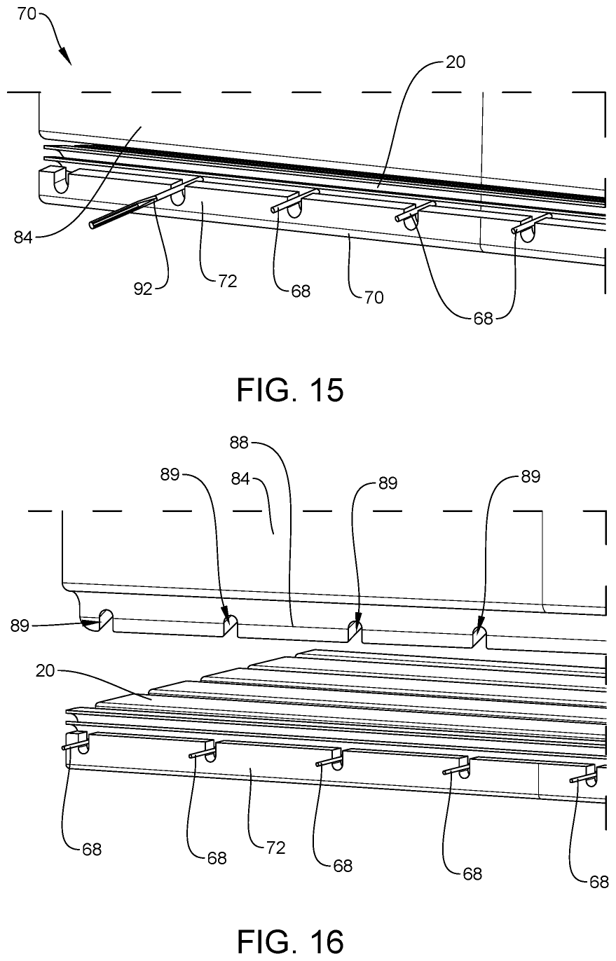

In some embodiments, suture 68 is inserted through only one of the series of holes 78 that extend through each of rails 74 and are coaxial with one another and a corresponding one of the series of grooves 89 that each extend through ribs 88 and are coaxial with one another along transverse axis X2. In some embodiments, suture 68 is inserted through one or more additional series of holes 78 that extend through each of rails 74 and are coaxial with one another and one or more additional one of the series of grooves 89 that each extend through ribs 88 and are coaxial with one another along transverse axis X2 after suture 68 is inserted through a first one of series of holes 78 that extend through each of rails 74 and are coaxial with one another and a first one of one of the series of grooves 89 that each extend through ribs 88 and are coaxial with one another along transverse axis X2. In some embodiments, suture 68 is inserted through each of the series of holes 78 that extend through each of rails 74 and are coaxial with one another and each of the one of the series of grooves 89 that each extend through ribs 88 and are coaxial with one another along transverse axis X2, as shown in FIGS. 15 and 16.

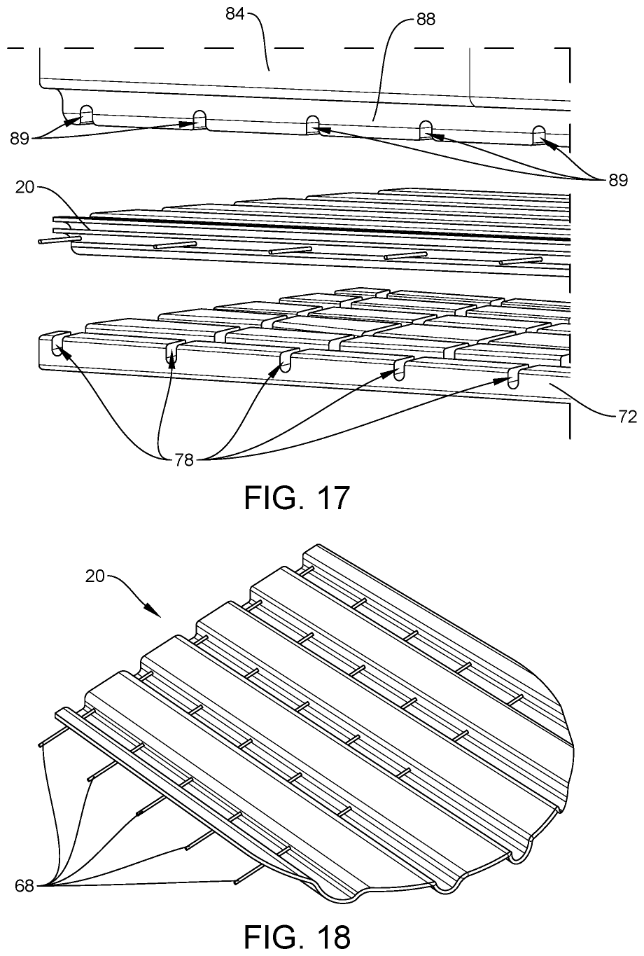

Once one or more sutures 68 have been inserted into anchorage device 20 in the manner discussed above, anchorage device 20 is removed from press 70 by removing plate 84 from anchorage device 20 and base 72 such that plate 84 is spaced apart from anchorage device 20 and base 72, as shown in FIG. 16. Anchorage device 20 is then moved in the direction shown by arrow D in FIG. 14 such that suture 68 moves out of holes 78 and through passageways 79. Anchorage device 20 is moved in the direction shown by arrow D in FIG. 14 until suture 68 moves passed surface 83 and anchorage device 20 is spaced apart from base 82, as shown in FIG. 17. In some embodiments, needle 92 has a diameter that is greater than diameters of passageways 79, such as, for example maximum diameters of passageways 79 and/or maximum uniform diameters of passageways 79 such that needle 92 is prevented from moving through passageways 79. In some embodiments, needle 92 has a diameter that is equal or substantially equal to diameters of passageways 79, such as, for example maximum diameters of passageways 79 and/or maximum uniform diameters of passageways 79 such that needle 92 is capable of being moved through passageways 79. An exemplary image of anchorage 20 after suture(s) 68 are inserted into anchorage device is shown in FIG. 18.

In some embodiments, anchorage device 20 is trimmed after the suture(s) is/are inserted into anchorage device 20 to join component 26 with component 22 in a manner that prevents component 26 from being removed from cavity 34 in a manner that is the same or similar to the in which anchorage device 20 is trimmed after seal(s) 62 is/are created by one or more heat seal bands 60 to join component 26 with component 22 in a manner that prevents component 26 from being removed from cavity 34.

In some embodiments, a needle, such as, for example, needle 92 is not used to suture component 26 with component 22 and an end of suture 68 is hardened as to allow the hardened end of suture 68 to be inserted through anchorage device 20 and openings 78 to thread suture 68 through components 22, 26 in the manner discussed above. It is further envisioned that alternative tools and/or instruments may be used in place of a needle to thread suture 68 through components 22, 26 in the manner discussed above.

In some embodiments, kits are provided that include one or a plurality of anchorage devices, such as, for example, anchorage devices 20. It is contemplated that each of the anchorage devices included can have a different configuration. In some embodiments, the anchorage devices can include different coatings 38 and/or 40. In some embodiments, the anchorage devices can include different sizes. In some embodiments, the anchorage devices can include different shapes. In some embodiments, the anchorage devices can include different anchorage devices that are designed for use with different medical devices, such as, for example, the implantable or non-implantable medical devices discussed herein. In some embodiments, the kits include one or a plurality of medical devices, such as, for example, the implantable or non-implantable medical devices discussed herein. In some embodiments, the kit includes instructions for use. In some embodiments, the kit includes items that are used to make the anchorage devices, such as, for example, the materials used to make the substrates, the hemostatic agent(s), the active pharmaceutical ingredient(s), a computer with a processor capable of receiving data and communicating with a 3D printer to create an anchorage device having the parameters that were input into the computer (e.g., size, shape, material, agents/ingredients on selected areas of the substrate in selected amounts) and a 3D printer capable of making the anchorage device based upon data that is input into the computer regarding the parameters of the implant.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method of forming an implant, the method comprising:
positioning a first mesh component of the implant within a second mesh component of the implant to form an implant assembly; and
manipulating the implant assembly to join the first mesh component with the second mesh component.

2. The method recited in claim 1, wherein manipulating the implant assembly comprises dispensing a plurality of stakes through the second mesh component and into the first mesh component.

3. The method recited in claim 2, wherein the stakes comprise collagen.

4. The method recited in claim 2, wherein the stakes comprise gelling collagen.

5. The method recited in claim 2, wherein the stakes are spaced apart from one another.

6. The method recited in claim 2, wherein the stakes are arranged in a pattern.

7. The method recited in claim 2, wherein each of the stakes is connected to another one of the stakes such that the stakes form a continuous line.

8. The method recited in claim 2, wherein the stakes extend about at least a portion of a perimeter of the implant assembly.

9. The method recited in claim 2, further comprising cooling the implant assembly after manipulating the implant assembly.

10. The method recited in claim 1, wherein manipulating the implant assembly comprises pressing an element of a heat seal band onto the implant assembly.

11. The method recited in claim 10, wherein the heat seal band forms a plurality of spaced apart horizontal seals across the implant assembly.

12. The method recited in claim 10, wherein the heat seal band forms a seal about at least a portion of a perimeter of the implant assembly.

13. The method recited in claim 10, wherein an interface is positioned between the heat seal band and the implant assembly to facilitate release of the heat seal band from the implant assembly after the implant assembly is manipulated.

14. The method recited in claim 1, wherein manipulating the implant assembly comprises using a first heat seal band to form a plurality of spaced apart horizontal seals across the implant assembly and using a second heat seal band to form a seal about at least a portion of a perimeter of the implant assembly after using the first heat seal band.

15. The method recited in claim 1, wherein manipulating the implant assembly comprises directing heat from a heat seal band onto the implant assembly to form at least one seal.

16. The method recited in claim 15, wherein an interface is positioned between the heat seal band and the implant assembly.

17. The method recited in claim 1, further comprising providing a press having a base, the base including a plurality of spaced apart rails that define channels therebetween, the base comprising a plurality of spaced apart holes extending through each of the rails, wherein manipulating the implant assembly comprises disposing the implant assembly in the base such that the implant assembly extends into the channels and inserting sutures through the holes and the implant assembly.

18. The method recited in claim 17, further comprising moving a plate of the press toward the base with the implant assembly positioned between the plate and the base to move portions of the implant assembly into the channels before inserting sutures through the holes and the implant assembly.

19. A method of forming an implant, the method comprising:
positioning a first mesh component of the implant within a second mesh component of the implant to form an implant assembly; and
manipulating the implant assembly to join the first mesh component with the second mesh component,
wherein the first mesh component comprises a coating having a first polymer and at least one antibacterial agent dispersed in the first polymer,
wherein the second mesh component comprises a coating having a second polymer and at least one hemostatic agent dispersed in the second polymer, and
wherein manipulating the implant assembly comprises dispensing a plurality of collagen stakes through the second mesh component and into the first mesh component.

20. A method of forming an implant, the method comprising:
positioning a first mesh component of the implant within a second mesh component of the implant to form an implant assembly; and
manipulating the implant assembly to join the first mesh component with the second mesh component,
wherein the first mesh component comprises a coating having a first polymer and at least one antibacterial agent dispersed in the first polymer,
wherein the second mesh component comprises a coating having a second polymer and at least one hemostatic agent dispersed in the second polymer, and
wherein manipulating the implant assembly comprises forming at least one seal by applying heat.
